(19) United States
(12) Patent Application Publication
(10) Pub. No.: US 2026/0345546 A1
(43) Pub. Date: Nov. 5, 2026

(54) COMPOUNDS FOR ENHANCEMENT OF MITOCHONDRIAL FUNCTION

(71) Applicant: Amazentis SA, Lausanne (CH)

(72) Inventors: Christopher L. Rinsch, Lausanne (CH); Davide D'Amico, Renens (CH)

(21) Appl. No.: 19/427,351

(22) Filed: Dec. 19, 2025

(30) Foreign Application Priority Data
Dec. 24, 2024 (GB) 2419076.1

(51) Int. Cl. A61K 31/352; A61K 31/05; A61K 31/366; A61K 31/404

(52) U.S. Cl. CPC A61K 31/352; A61K 31/05; A61K 31/366; A61K 31/404

(57) ABSTRACT

The present invention relates to novel uses of compounds as enhancers of mitochondrial functions. The invention further relates to use of such mitochondrial function enhancers for the treatment of diseases, disorders and conditions and for health improvements in generally healthy individuals.

### RELATED APPLICATION

This application claims the benefit of priority to UK Patent Application No. GB 2419076.1, filed Dec. 24, 2024.

### BACKGROUND

Mitochondria are the energy plant of cells, and healthy mitochondria are like a power plant that converts energy through the Krebs cycle and oxidative phosphorylation. Mitochondrial function can determine the survival of cells, on the one hand, the production of ATP by oxidative phosphorylation to provide energy requirements for cells, and the transport of calcium ions in the cytoplasm to mitochondrial storage through calcium channels, enabling cells to maintain normal function; Abnormal accumulation of mitochondrial intracellular reactive oxygen species (ROS) and the production of apoptosis-related proteins, such as cytochrome c and apoptosis-inducing factors, cause cell damage and induce cell death. In addition, mitochondria have a separate gene coding system, and mitochondrial DNA mutations due to aging or damage are not easily repaired due to the specificity of mitochondrial gene organization. Given these key factors, damaged mitochondria can cause damage to cells, and dysfunction of mitochondria can lead to many types of diseases, including heart failure, Alzheimer's disease, and cancer.

Mitophagy is a process in which cells selectively remove damaged or senescent mitochondria and recycle their constituent elements. Mitophagy balances the quality and quantity of mitochondria in cells and is therefore important for maintaining cell homeostasis and cell survival. Studies have shown that mitophagy has obvious protective effects in different pathogenic factors and different cells, and its defects and dysfunction are closely related to many physiological and pathological processes such as aging, neurodegenerative diseases and cancer. Recently, more and more studies have shown that mitophagy is also involved in the development of various liver diseases, for example, non-alcoholic fatty liver, liver fibrosis/cirrhosis, liver cancer and hepatic ischemia-reperfusion injury.

It has surprisingly been found that a number of compounds are enhancers of mitophagy and mitochondrial function, which opens up new possibilities for the treatment of diseases, disorders and conditions, and possibilities for enhancing health in generally healthy individuals, such as promoting general health, muscle function and healthy aging.

### SUMMARY

The present invention relates to compounds which are enhancers of mitochondrial functions. The invention further relates to use of such mitochondrial function enhancers for the treatment of diseases, disorders and conditions and for health improvements in generally healthy individuals.

Therefore, according to a first embodiment of the invention there is provided a compound of Table 1 or Table 1a, or a salt, derivative or prodrug thereof, for use in enhancing or maintaining mitochondrial function.

In a further aspect of the invention, there is provide a compound of Table 1 or Table 1a, or a salt, derivative or prodrug thereof, for increasing or maintaining mitochondrial function.

In a further embodiment of the invention there is provided a compound of Table 1 or Table 1a, or a salt, derivative or prodrug thereof, for use in a method of enhancing mitophagy.

In a further embodiment of the invention, there is provided a compound of Table 1 or Table 1a, or a salt, derivative or prodrug thereof, for the treatment of a disease, disorder or condition associated with a decrease in mitophagy.

In a further embodiment of the invention, there is provided a compound of Table 1 or Table 1a, or a salt, derivative or prodrug thereof, for administration to a generally healthy subject who will benefit from an increase in mitophagy.

In a further embodiment of the invention, there is provided a compound of Table 1 or Table 1a, or a salt, derivative or prodrug thereof, for use in a method of enhancing mitophagy in a generally healthy subject.

In a further embodiment of the invention, there is provided a compound of Table 1 or Table 1a, or a salt, derivative or prodrug thereof, inhibitor for treatment, prevention or management of a mitochondria-related condition associated with altered or diminished mitochondrial function or reduced mitochondrial density.

In a further embodiment of the invention, there is provided a compound of Table 1 or Table 1a, or a salt, derivative or prodrug thereof, for use in the treatment, prevention or management of a mitochondria-related disease or condition associated with an altered mitochondrial function or a reduced mitochondrial density.

In a further embodiment of the invention, there is provided a compound of Table 1 or Table 1a, or a salt, derivative or prodrug thereof, for use in the treatment, prevention or management of conditions, diseases, and disorders related to mitochondrial dysfunction.

In a further embodiment of the invention, there is provided a compound of Table 1 or Table 1a, or a salt, derivative or prodrug thereof, for use in the treatment, prevention of a disease initiated or characterized by inadequate mitochondrial function.

In a further embodiment of the invention, there is provided a compound of Table 1 or Table 1a, or a salt, derivative or prodrug thereof, for use to increase mitochondrial activity, to increase mitochondrial biogenesis, and/or to increase mitochondrial mass.

In a further embodiment of the invention, there is provided a compound of Table 1 or Table 1a, or a salt, derivative or prodrug thereof, for use in the treatment, prevention or management of diseases and conditions in which defective or diminished mitochondrial activity participates in the pathophysiology of the disease or condition, or in which increased mitochondrial function will yield a desired beneficial effect.

In a further embodiment of the invention, there is provided a compound of Table 1 or Table 1a, or a salt, derivative or prodrug thereof, for use in the treatment, prevention of a mitochondrial disease.

### BRIEF DESCRIPTION OF THE FIGURES

FIG. 1A: Impact of test compounds (Examples 1 to 4) on maximal respiration in C2C12 mouse myotubes, measured as oxygen consumption rate (OCR; pmol/min).

FIG. 1B: Impact of test compounds (Examples 5 to 8) on maximal respiration in C2C12 mouse myotubes, measured as oxygen consumption rate (OCR; pmol/min).

FIG. 1C: Impact of test compounds (Examples 9 to 11) on maximal respiration in C2C12 mouse myotubes, measured as oxygen consumption rate (OCR; pmol/min).

FIG. 2: Relative gene expression in human dermal fibroblasts upon treatment with indicated doses of Norartocarpetin.

FIG. 3: Prostaglandin E2 (PGE2) release (pg/ml) in human dermal keratinocyte cultures following UV treatment, with or without Norartocarpetin exposure, compared to non-UV controls.

FIG. 4: Quantification of actin depolymerization in human dermal fibroblasts comparing young donor, old donor and old donor treated with Norartocarpetin.

FIG. 5A: Representative images of actin in human dermal fibroblasts as in FIG. 4. A: young donor.

FIG. 5B: Representative images of actin in human dermal fibroblasts as in FIG. 4. B: old donor.

FIG. 5C: Representative images of actin in human dermal fibroblasts as in FIG. 4. C: old donor treated with Norartocarpetin.

FIG. 6A: Quantification of nuclear perimeter (A) in human dermal fibroblasts comparing young donor, old donor and old donor treated with Norartocarpetin.

FIG. 6B: Quantification of nuclear area (B) in human dermal fibroblasts comparing young donor, old donor and old donor treated with Norartocarpetin.

FIG. 6C: Quantification of nuclear circularity (C) in human dermal fibroblasts comparing young donor, old donor and old donor treated with Norartocarpetin.

FIG. 7: Maximal mitochondrial respiration in C2C12 mouse myotubes, measured as oxygen consumption rate (OCR; pmol/min), comparing treatment with Norartocarpetin alone, resveratrol alone and a combination of both Norartocarpetin and resveratrol.

### DETAILED DESCRIPTION

Disclosed herein is a composition comprising one or more compounds selected from: norartocarpetin, juglanin, meisoindigo, byakangelicol, n-nornuciferine, 3′-demethylnobiletin, 3-o-methylquercetin, dehydrocorydaline (hydroxyl), pachypodol, 4′,5-dihydroxyflavone, 5-hydroxyflavone, scutellarein tetramethyl ether, isosinensetin, robustine, skullcapflavone II, dehydrocorydaline (nitrate), coptisine (for example, the chloride salt), mosloflavone, iristectorin B, chrysosplenol d, phellopterin, chrysosplenetin, angelicin, and dehydrocorydaline (for example, the chloride salt), for use in a method of enhancing mitophagy.

In some embodiments, a composition as disclosed herein for the treatment of a disease, disorder or condition associated with a decrease in mitophagy, or for use in a method of enhancing mitophagy.

In some embodiments, a composition as disclosed herein for use in a method of enhancing mitophagy in a generally healthy subject.

In some embodiments, wherein the use enhances or maintains mitochondrial function.

In some embodiments, wherein the disease, disorder or condition is associated with altered mitochondrial function or reduced mitochondrial density.

In some embodiments, wherein the use increases mitochondrial activity, increases mitochondrial biogenesis, and/or increases mitochondrial mass.

In some embodiments, a composition as disclosed herein for enhancing muscle performance, for improving endurance capacity, or for improving, maintaining or reducing the loss of muscle function in a subject.

In some embodiments, wherein the subject suffers physical fatigue, and/or muscle fatigue.

In some embodiments, wherein the subject the subject is frail or pre frail.

In some embodiments, a composition as disclosed herein for enhancing immune health, and/or slow inflammaging.

In some embodiments, a composition as disclosed herein for maintaining or enhancing brain health.

In some embodiments, a composition as disclosed herein for treating or preventing skin conditions, treating hair loss and/or hair thinning.

In some embodiments, a composition as disclosed herein for increasing healthspan and/or promoting healthy aging.

In some embodiments, a composition as disclosed herein for use in the treatment of a muscle-related pathological condition, for example, wherein the muscle-related pathological condition is selected from musculoskeletal diseases or disorders; muscle wasting; myopathies; neuromuscular diseases, such as Duchenne muscular dystrophy and other dystrophies sarcopenia, for example, acute sarcopenia; and muscle atrophy and/or cachexia, for example muscle atrophy and/or cachexia associated with burns, bed rest, limb immobilization, or major thoracic, abdominal, and/or orthopaedic surgery.

In some embodiments, a composition as disclosed herein for use in the treatment of neurological disorders, including neurodegenerative disorders.

In some embodiments, wherein the disorder is selected from cognitive decline, memory decline, anxiety, depression, epilepsy, stroke, amyotrophic lateral sclerosis, dementia (e.g. vascular dementia and Alzheimer's disease), and Huntington disease.

In some embodiments, a composition as disclosed herein for use in enhancing immune function.

In some embodiments, wherein the compound is selected from Norartocarpetin, I, Juglanin, Meisoindigo, Byakangelicol, 3′-Demethylnobiletin, 3-O-Methylquercetin, Pachypodol, 5-Hydroxyflavone, Scutellarein tetramethyl ether, Isosinensetin, (for example, the chloride salt).

In some embodiments, wherein the compound is selected from N-Nornuciferine, Dehydrocorydaline (hydroxyl), 4′,5-Dihydroxyflavone, Robustine, Skullcapflavone II, Dehydrocorydaline (for example, the nitrate salt), Coptisine (for example, the chloride salt), Mosloflavone, Iristectorin B, Chrysosplenol D, Phellopterin, Chrysosplenetin, Angelicin, Dehydrocorydaline (for example, the chloride salt).

In some embodiments, a nutraceutical composition comprising one of more compounds of the composition disclosed herein.

In some embodiments, a cosmetic composition comprising one of more compounds the composition disclosed herein.

In some embodiments, a pharmaceutical composition comprising one of more compounds the composition disclosed herein.

Mitochondrial diseases include, without limitation, Alper's disease; Barth syndrome; beta-oxidation defects; carnitine deficiency; carnitine-acyl-carnitine deficiency; chronic progressive external ophthalmoplegia syndrome; co-enzyme Q10 deficiency; Complex I deficiency; Complex II deficiency; Complex III deficiency; Complex IV deficiency; Complex V deficiency; CPT I deficiency; CPT II deficiency; creatine deficiency syndrome; cytochrome c oxidase deficiency; glutaric aciduria type II; Keams-Sayre syndrome; lactic acidosis; TCHAD (long-chain acyl-CoA dehydrogenase deficiency); Leber's hereditary optic neuropathy; Leigh disease; lethal infantile cardiomyopathy; Luft disease; MAD (medium-chain acyl-CoA dehydrogenase deficiency); mitochondrial cytopathy; mitochondrial DNA depletion; mitochondrial encephalomyopathy, lactic acidosis, and stroke-like symptoms; mitochondrial encephalopathy; mitochondrial myopathy; mitochondrial recessive ataxia syndrome; muscular dystrophies, myoclonic epilepsy and ragged-red fibre disease; myoneurogenic gastrointestinal encephalopathy; neuropathy, ataxia, retinitis pigmentosa, and ptosis; Pearson syndrome; POLG mutations; pyruvate carboxylase deficiency; pyruvate dehydrogenase deficiency; SCHAD (short-chain acyl-CoA dehydrogenase deficiency); and very long-chain acyl-CoA dehydrogenase deficiency.

In a further embodiment of the invention, there is provided a compound of Table 1 or Table 1a, or a salt, derivative or pro-drug thereof, for a mitochondrial use as defined above, with the proviso that the following compound are excluded: chrysoeriol,

In methods and uses of the invention, the reference to a compound of Table 1 or Table 1a, or a salt, derivative or pro-drug thereof, also refers to a combination of one, two, three of four compounds of Table 1 or Table 1a, or salts, derivatives or prodrugs thereof.

In a preferred embodiment of the invention, a compound of Table 1 or Table 1a is selected from Norartocarpetin, Chrysoeriol, Juglanin, Meisoindigo, Byakangelicol, 3′-Demethylnobiletin, 3-O-Methylquercetin, Pachypodol, 5-Hydroxyflavone, Scutellarein tetramethyl ether, Isosinensetin, (for example, the chloride salt).

In a further preferred embodiment of the invention, a compound of Table 1 or Table 1a is selected from N-Nornuciferine, Dehydrocorydaline (hydroxyl), 4′,5-Dihydroxyflavone, Robustine, Skullcapflavone II, Dehydrocorydaline (for example, the nitrate salt), Coptisine (for example, the chloride salt), Mosloflavone, Iristectorin B, Chrysosplenol D, Phellopterin, Chrysosplenetin, Angelicin, Dehydrocorydaline (for example, the chloride salt).

TABLE 1

| Example | Structure | Compound Name |
|---|---|---|
| 1 | 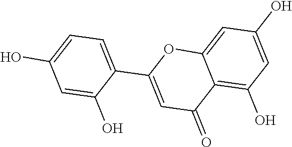 [SMILES: O=c1cc(-c2ccc(O)cc2O)oc2cc(O)cc(O)c12] | Norartocarpetin |
| 2 | 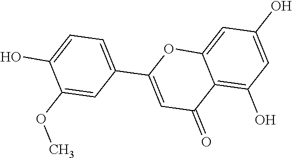 [SMILES: COc1cc(-c2cc(=O)c3c(O)cc(O)cc3o2)ccc1O] | Chrysoeriol |
| 3 | 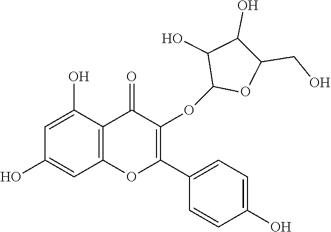 [SMILES: O=c1c(OC2OC(CO)C(O)C2O)c(-c2ccc(O)cc2)oc2cc(O)cc(O)c12] | Juglanin |
| 4 | 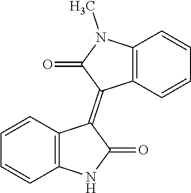 [SMILES: CN1C(=O)/C(=C2/C(=O)Nc3ccccc32)c2ccccc21] | Meisoindigo |
| 5 | 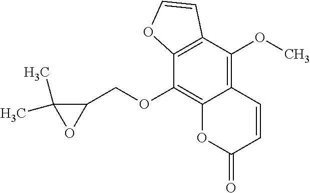 | Byakangelicol |
| 6 | 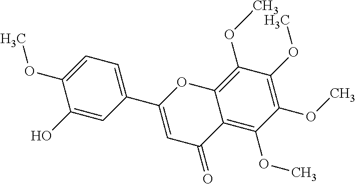 | 3′-Demethylnobiletin |
| 7 | 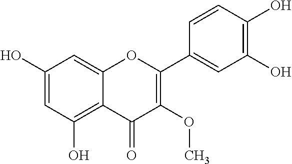 [SMILES: COc1c(-c2ccc(O)c(O)c2)oc2cc(O)cc(O)c2c1=O] | 3-O-Methylquercetin |
| 8 | 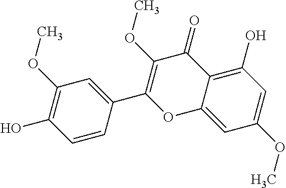 [SMILES: COc1cc(O)c2c(=O)c(OC)c(-c3ccc(O)c(OC)c3)oc2c1] | Pachypodol |
| 9 | 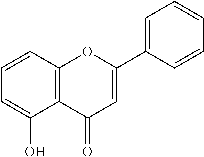 [SMILES: O=c1cc(-c2ccccc2)oc2cccc(O)c12] | 5-Hydroxyflavone |
| 10 | 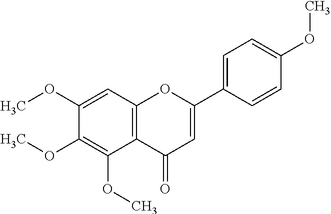 | Scutellarein tetramethyl ether |
| 11 | 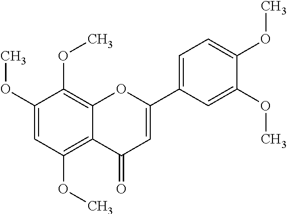 [SMILES: COc1ccc(-c2cc(=O)c3c(OC)cc(OC)c(OC)c3o2)cc1OC] | Isosinensetin |
| 12 | 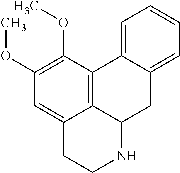 [SMILES: COc1cc2c3c(c1OC)-c1ccccc1CC3NCC2] | N-Nornuciferine |
| 13 | 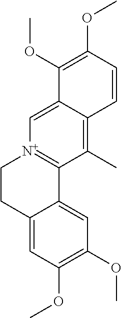 [SMILES: COc1cc2c(cc1OC)-c1c(C)c3ccc(OC)c(OC)c3c[n+]1CC2] | Dehydrocorydaline (hydroxyl) |
| 14 | 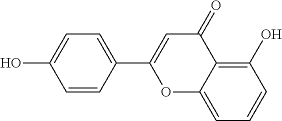 [SMILES: O=c1cc(-c2ccc(O)cc2)oc2cccc(O)c12] | 4′,5-Dihydroxyflavone |
| 15 | 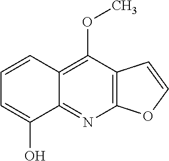 [SMILES: COc1c2ccoc2nc2c(O)cccc12] | Robustine |
| 16 | 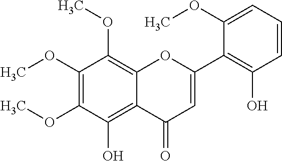 [SMILES: COc1cccc(O)c1-c1cc(=O)c2c(O)c(OC)c(OC)c(OC)c2o1] | Skullcapflavone II |
| 17 | 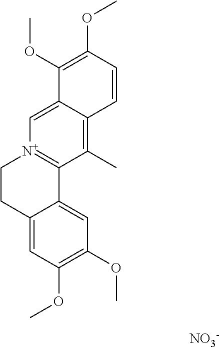 [SMILES: COc1cc2c(cc1OC)-c1c(C)c3ccc(OC)c(OC)c3c[n+]1CC2 ; "NO3-"] | Dehydrocorydaline (nitrate) |
| 18 | 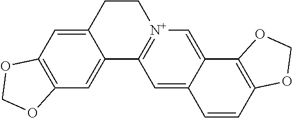 [SMILES: c1c2c(cc3c1OCO3)-c1cc3ccc4c(c3c[n+]1CC2)OCO4] | Coptisine (chloride) |
| 19 | 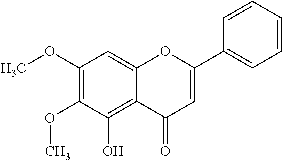 [SMILES: COc1cc2oc(-c3ccccc3)cc(=O)c2c(O)c1OC] | Mosloflavone |
| 20 | 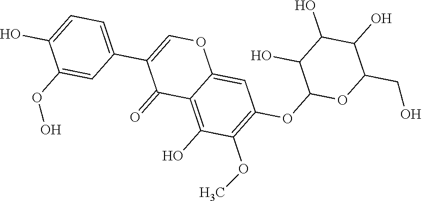 [SMILES: COc1c(OC2OC(CO)C(O)C(O)C2O)cc2occ(-c3ccc(O)c(OO)c3)c(=O)c2c1O] | Iristectorin B |
| 21 | 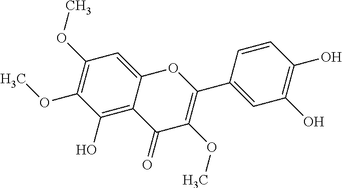 [SMILES: COc1cc2oc(-c3ccc(O)c(O)c3)c(OC)c(=O)c2c(O)c1OC] | Chrysosplenol D |
| 22 | 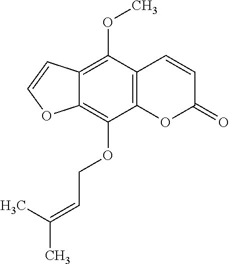 [SMILES: COc1c2ccoc2c(OCC=C(C)C)c2oc(=O)ccc12] | Phellopterin |
| 23 | 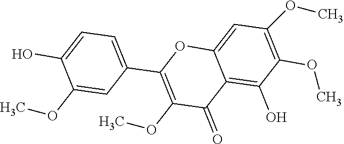 | Chrysosplenetin |
| 24 | 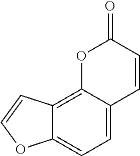 [SMILES: O=c1ccc2ccc3occc3c2o1] | Angelicin |
| 25 | 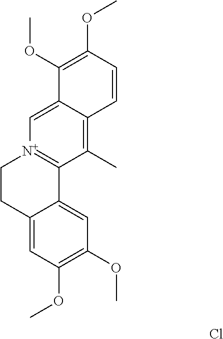 [SMILES: COc1cc2c(cc1OC)-c1c(C)c3ccc(OC)c(OC)c3c[n+]1CC2 ; Cl] | Dehydrocorydaline (chloride) |

TABLE 1a

| Example | Structure | Compound Name |
|---|---|---|
| 1 | 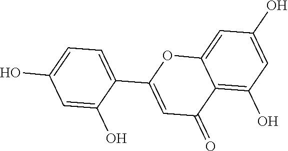 [SMILES: O=c1cc(-c2ccc(O)cc2O)oc2cc(O)cc(O)c12] | Norartocarpetin |
| 2 | 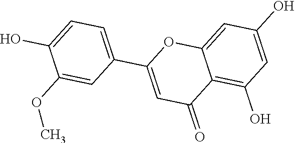 [SMILES: COc1cc(-c2cc(=O)c3c(O)cc(O)cc3o2)ccc1O] | Chrysoeriol |
| 3 | 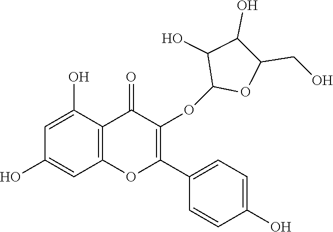 [SMILES: O=c1c(OC2OC(CO)C(O)C2O)c(-c2ccc(O)cc2)oc2cc(O)cc(O)c12] | Juglanin |
| 6 | 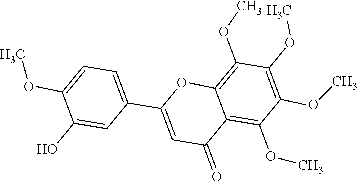 | 3′-Demethylnobiletin |
| 7 | 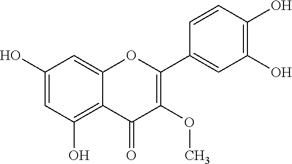 [SMILES: COc1c(-c2ccc(O)c(O)c2)oc2cc(O)cc(O)c2c1=O] | 3-O-Methylquercetin |
| 8 | 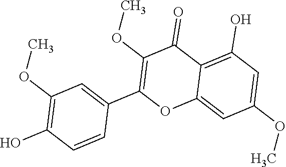 [SMILES: COc1cc(O)c2c(=O)c(OC)c(-c3ccc(O)c(OC)c3)oc2c1] | Pachypodol |
| 10 | 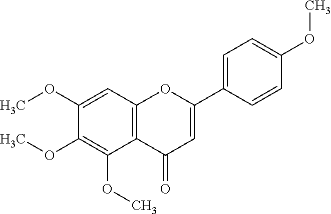 | Scutellarein tetramethyl ether |
| 11 | 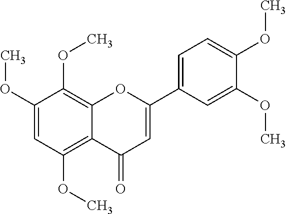 [SMILES: COc1ccc(-c2cc(=O)c3c(OC)cc(OC)c(OC)c3o2)cc1OC] | Isosinensetin |
| 14 | 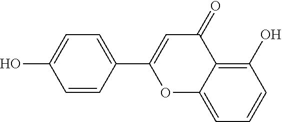 [SMILES: O=c1cc(-c2ccc(O)cc2)oc2cccc(O)c12] | 4′,5-Dihydroxyflavone |
| 16 | 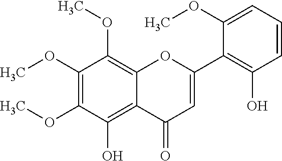 [SMILES: COc1cccc(O)c1-c1cc(=O)c2c(O)c(OC)c(OC)c(OC)c2o1] | Skullcapflavone II |
| 19 | 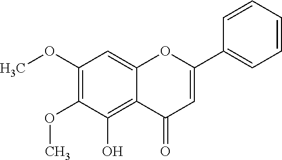 [SMILES: COc1cc2oc(-c3ccccc3)cc(=O)c2c(O)c1OC] | Mosloflavone |
| 20 | 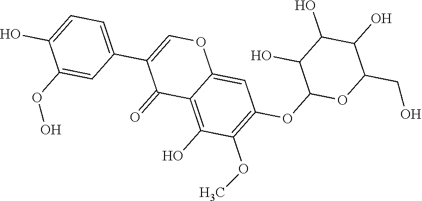 [SMILES: COc1c(OC2OC(CO)C(O)C(O)C2O)cc2occ(-c3ccc(O)c(OO)c3)c(=O)c2c1O] | Iristectorin B |
| 21 | 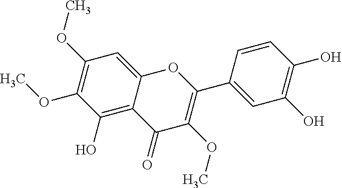 [SMILES: COc1cc2oc(-c3ccc(O)c(O)c3)c(OC)c(=O)c2c(O)c1OC] | Chrysosplenol D |
| 23 | 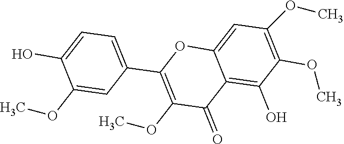 | Chrysosplenetin |

Compounds of Table 1 or Table 1a also comprise compositions with different levels of purity, including having an origin of natural extracts containing them.

The identification of compounds of Table 1 or Table 1a as enhancer of mitochondrial activity opens up a number or new possibilities for the treatment of disease, disorders and conditions, and for use in improving health and fitness in generally healthy individuals. Such uses (applications) are listed in Table 2. In a further embodiment, there is provided a compound of Table 1 or Table 1a, or a salt, derivative or prodrug thereof, for a use listed in column 2 of Table 2. In a further embodiment, there is provided a compound of Table 1 or Table 1a, or a salt, derivative or prodrug thereof, for a use listed in column 3 of Table 2.

In Table 2 below, references to ‘supports’ also refers to helps, helps support, improves, enhances, increases, maintains, boosts, restores, optimises and/or promotes, and references to other words in this list, also refer to each other word, for example, ‘enhances’ also refers to supports, helps, helps support, improves, increases, maintains, boosts, restores, optimises and/or promotes.

In Table 2 below, references to ‘reverses’ also refers to reduces, slows, protects, manage, inhibits and/or prevents, and references to other words in this list, also refer to each other word.

TABLE 2

| | | Applications for |
|---|---|---|
| Health Benefit | Applications for Healthy Persons | Disease States |
| Aging/Health Span/ | Anti-aging | |
| Life Span/ Healthy | Supports Healthy Aging | |
| Aging | Support healthy cellular aging | |
| | Supports overall wellness and | |
| | longevity | |
| | Improves health-span or healthspan | |
| Improved Cellular | Stimulates Cellular Energy | Tumour suppression |
| Health | Maintains cellular energy | |
| | prevents age-associated cellular | |
| | decline | |
| | reverses biological age | |
| Mitophagy/autophagy/ | Improves the quality of mitochondria | |
| lysophagy | increases mitochondrial recycling | |
| | Increases mitophagy | |
| | Increases lysophagy | |
| | Increases autophagy | |
| Metabolism | Maintenance of the amino acid pool | Reduction of symptoms |
| | during starvation | metabolic disorders, |
| | | diabetes and obesity |
| | Protects Against Oxidative Stress | Improved outcome in |
| | | acute pancreatitis |
| | Helps reduce oxidative stress | |
| | Improves cellular metabolism | |
| | Helps reduce muscle soreness from | |
| | normal daily activities | |
| | reverses insulin resistance | beneficial for pre- |
| | | diabetics, diabetics |
| Mitochondria | Improves mitochondrial efficiency | prevention or treatment |
| | | of disease symptoms |
| | | caused by or |
| | | characterized by |
| | | mitochondrial |
| | | hypofunction |
| | Maintain, Improve cellular energy | |
| | Improved bioenergetics | |
| | Supports energy metabolism | |
| | Supports mitochondrial, cellular and | |
| | whole body health | |
| Brain | prevents cognitive decline with aging | Reduction of symptoms |
| | | neurodegenerative |
| | | diseases, cognitive |
| | | disorders, mood |
| | | disorders, anxiety |
| | | disorders |
| | nootropic effect | Prevention of |
| | | neurodegeneration |
| | enhances neurotransmission | Clearance of protein |
| | | aggregates |
| | Enhancement of cognitive function | Alzheimer′s Disease, |
| | | ALS and other |
| | | neurodegenerative and |
| | | mood related disorders |
| | increased mitochondrial activity to | Frontotemporal |
| | improve brain function, | Dementia, Lewy Body |
| | | Dementia, Multiple |
| | | System Atrophy, |
| | | Progressive |
| | | Supranuclear Palsy, |
| | | Spinocerebellar Ataxias, |
| | | Prion Diseases |
| | | (including Creutzfeldt- |
| | | Jakob Disease), Major |
| | | Depressive Disorder, |
| | | Bipolar Disorder, |
| | | Generalized Anxiety |
| | | Disorder, Schizophrenia, |
| | | Post-Traumatic Stress |
| | | Disorder, Epilepsy, |
| | | Migraine, Chronic Pain |
| | | Syndromes, Autism |
| | | Spectrum Disorders, |
| | | Obsessive-Compulsive |
| | | Disorder |
| | Supports Brain Health and Cognitive | |
| | Function | |
| | Supports psychomotor speed, | |
| | reaction time | |
| | Helps support cognitive health | |
| | Supports learning | |
| | Supports memory | |
| | Supports mental focus | |
| | Supports accuracy | |
| | Supports mental processing | |
| | Supports cognitive function, | |
| | performance | |
| | Supports Attention/Concentration | |
| | Supports concentration | |
| | Supports attention and cognitive | |
| | function | |
| | Support complex and sustained | |
| | attention | |
| | Supports Healthy Stress Management | |
| | Supports Healthy Mood | |
| | Supports mood | |
| | Supports healthy stress and | |
| | occasional blues | |
| | Supports healthy mood and quality of | |
| | life | |
| | Supports Healthy Sleep Quality | |
| | Supports cognitive health | |
| | Supports healthy brain function | |
| | Supports your ability to deal with | |
| | mental and physical stress | |
| Skeletal Muscle | improves muscle performance and | Reduction in sarcopenia |
| | mobility | |
| | improves physical performance | Improved outcome for |
| | | muscular dystrophy |
| | | Spinal muscular atrophy |
| | | (SMA) and inclusion |
| | | body myositis (IBM) |
| | Improves skeletal muscle functions | |
| | Promotes muscle energy | |
| | Improved balance and coordination | |
| | Improvement in muscle strength, e.g. | |
| | first interosseous muscle | |
| | Increase in muscle mass | |
| | Reduction in muscle atrophy | |
| | Improvement in muscle endurance | |
| | Improves muscle resistance to fatigue | |
| | Improve muscle recovery | |
| | Improvement in muscle recovery | |
| | following exercise | |
| | Protection against muscle damage | |
| | reduces muscle damage following | |
| | exercise | |
| | may improve muscle strength and | |
| | size when used in conjunction with | |
| | regular resistance training | |
| Heart muscle | Supports heart health and energy | Improvement outcome |
| | production | from |
| | | ischemia/reperfusion |
| | | injury |
| | | Protection of cells from |
| | | ischemia and hypoxic |
| | | conditions resulting from |
| | | myocardial function |
| | Supports cardiovascular health | May reduce the risk of |
| | | heart disease |
| | Improves diastolic function | Improved outcome in |
| | | various heart conditions |
| | | such as heart disease, |
| | | cardiac hypertrophy, left |
| | | ventricular dilation, |
| | | Danon disease |
| | Improves systolic function | Amelioration of |
| | | cardiomyopathy resulting |
| | | from diabetes |
| | Increases heart contractility | Protection of cells during |
| | | cardiac procedures such |
| | | as angioplasty, bypass, |
| | | valve replacement |
| | Increases heart energy availability | |
| | Improved cardiac output | |
| | Improvement in blood pressure | |
| | Preventing cardiac deterioration with | |
| | age | |
| Immune | Immune Support | |
| | Increases mitochondrial function in | |
| | immune cells, | |
| | increases the population of immune | |
| | cells, for example, T cells | |
| | Supports Immune System Health | |
| | Supports T-cell populations important | Protects against |
| | for immune response (increase naïve | autoimmune diseases |
| | cells) | |
| | Increases natural killer cells important | protects against sepsis |
| | in the innate immune response | |
| | Increase in non-classical monocytes, | Clearance of intracellular |
| | more anti-inflammatory | microbes |
| | Decreases markers of immune aging, | |
| | reduces immune aging | |
| | Induces immune re-modelling | |
| | Improved Mitochondrial health of | |
| | immune cells | |
| | Improves mitochondrial function of | |
| | CD8 T cells, key effector cells of the | |
| | immune system | |
| | Improves mitochondrial function in | |
| | immune cells important for immune | |
| | response | |
| | Global rewiring of the immune system | |
| | to a more youthful and functional | |
| | immune system | |
| | Slowing immune aging, attenuates | |
| | plasma cytokines markers of | |
| | inflammaging in healthy adults | |
| | Slows immune aging | |
| | Improved Immune Function | |
| | Supports Immune Function | |
| | Important for proper immune function | |
| | Improve mitochondria density in your | |
| | immune cells | |
| | Improve mitochondria function in your | |
| | immune cells after one month | |
| | Reduces systemic inflammation | |
| | Supports Vitality and Immune | |
| | Function | |
| | Support for Immune Health | |
| | boosts immunotherapy responses | |
| | boosts vaccine antibody responses | |
| Skin | Supports Healthy Skin | protects against |
| | | psoriasis |
| | Improves cells′ structure to activate | reduces atopic dermatitis |
| | skin′s adaptive responses to daily | |
| | environmental conditions. | |
| | Increases mitochondrial function in | |
| | skin cells including dermal fibroblasts, | |
| | keratinocytes, melanocytes, | |
| | sebocytes, Langerhans cells | |
| | Reduces skin phototoxicity caused by | |
| | UV irradiation | |
| | Help to reduce skin dryness from | |
| | within | |
| | Rebuilding skin structure: increasing | |
| | the quality and thickness/density of | |
| | skin | |
| | Clinically proven to reach all skin′s | |
| | layer targeting immediate skin | |
| | concerns or delivering immediate | |
| | hydration, while progressively adding | |
| | skin health from within and improve | |
| | skin conditions overtime or | |
| | strengthening skin for preventing. | |
| | Help regain skin substance, volume | |
| | and plumpness. | |
| | Improve skin elasticity. | |
| | Reduce appearance of wrinkles and | |
| | smooth lines | |
| | Stimulates collagen and elastin | |
| | production to enhance skin′s | |
| | smoothness | |
| | Detoxifies skin cells for youthful- | |
| | looking skin | |
| | Promote optimal skin health and | |
| | appearance. | |
| | Enhance skin radiance for a luminous | |
| | and vibrant complexion/glow | |
| | Holistic protocol from inside out | |
| | clinically proven to reverse skin | |
| | biological age | |
| | Clinically proven to reach all skin′s | |
| | layer from in and out to deliver | |
| | complementary cellular health | |
| | benefits for overall /holistic skin | |
| | health. | |
| | Clinically proven to reach all skin′s | |
| | layer targeting immediate skin | |
| | concerns or delivering immediate | |
| | hydration, while progressively adding | |
| | skin health from within and improve | |
| | skin conditions overtime or | |
| | strengthening skin for preventing. | |
| | Clinically proven to reduce damages | |
| | from outside and strengthen the skin | |
| | cells, preventing damages from | |
| | inside. | |
| | Clinically proven to protect and | |
| | strengthen skin cell barriers against | |
| | oxidation caused by external factors | |
| | (UV rays, pollutants) and increase the | |
| | skin′s ability to heal from inside. | |
| | Clinically proven to provide internal | |
| | support delivering specific nutriments | |
| | that could not permeate the skin for a | |
| | completely nourished skin | |
| | reduces aging skin spots | |
| | reverses skin biological age | |
| | protects against skin inflammation | eczema, acne, psoriasis |
| | conditions | |
| | Reduces inflammation in subjects | |
| | prone to skin inflammatory conditions, | |
| | e.g. psoriasis and dermatitis | |
| | protecting skin from damage caused | |
| | by pollution or radiation, e.g. UV, α, β | |
| | or γ radiation | |
| Hair | protecting hair from damage caused | |
| | by pollution or radiation, e.g. UV, α, β | |
| | or γ radiation | |
| | maintaining or enhancing hair | |
| | thickness | |
| | Enhancing hair follicle cell | |
| | regeneration | |
| | Enhancing hair follicle cell survival | |
| | Enhancing hair stem cell growth | |
| | and/or regeneration | |
| | Enhancing hair cell survival | |
| | Enhancing hair loss prevention | |
| | hair maintenance | |
| | scalp health improvement | |
| | improving or maintaining hair strength | |
| | Restores hair growth | |
| | Hair growth restoration | |
| | enhancing hair stem cell function | |
| | enhancing hair follicle elongation | |
| | enhancing hair matrix proliferation | |
| | enhancing hair growing phase, | |
| | (enhancing the duration of the | |
| | anaphase) | |
| | Enhancing hair matrix proliferation | |
| | inhibition of hair matrix apoptosis | |
| | reducing melanin clumping | |
| Gut | Improves gut health | improves inflammation |
| | | bowel disease |
| | | phenotypes |
| | Boosts good gut bacteria | improves gut diseases |
| | Support healthy microbiome | |
| | Increases butyrate production | |
| | improves bowel motility | constipation, IBS, IBD, |
| | | short bowel syndrome |
| | support digestion | |
| | reduces gut inflammation | |
| | Reduces leaky gut | |
| | boosts gut stem cell function | |
| Eye | Supports eye health | Improved outcome from |
| | | intraocular inflammation |
| | | or uveitis |
| | Improves vision | Reduction in |
| | | photoreceptor injury from |
| | | uveitis |
| | Maintaining or enhancing eye | |
| | epithelial cell health and/or function, | |
| | for example, corneal epithelial cells | |
| | Maintaining or enhancing cone and/or | |
| | rod cell health and/or function | |
| | Maintaining or enhancing lung cell | |
| | health and/or function, for example, | |
| | photoreceptor cells (for example rods | |
| | and/or cones), bipolar cells, ganglion | |
| | cells, horizontal cells, amacrine cells | |
| | glial cells (for example, Muller cells) | |
| | and/or epithelial cells | |
| | Supports eye cell function, including | |
| | retinal pigment epithelial cells, | |
| | photoreceptors, ganglion cells, | |
| | corneal epithelial cells, lens epithelial | |
| | cells, and Muller glial cells | |
| | Prevents age-related vision decline | Reduction in |
| | | glaucomatous |
| | | neurodegeneration |
| | Sharpens vision | Reduction in age related |
| | | macular degeneration |
| | | Protection from diabetic |
| | | retinopathy |
| | | Reduction in age-related |
| | | cataracts |
| Lung | Improves lung health (vital capacity, | Improved outcome from |
| | FEV1) | pulmonary emphysema |
| | | cause by α1-antrypsin |
| | Maintaining or enhancing lung | |
| | epithelial cell health and/or function | |
| | Maintaining or enhancing lung cell | |
| | health and/or function, for example, | |
| | cells selected from epithelial cells | |
| | (ciliated, goblet, basal, club/Clara, | |
| | neuroendocrine, ionocyte and/or Type | |
| | I & II alveolar cells), fibroblasts and/or | |
| | endothelia cells | |
| | Supports function of lung cells, | |
| | including alveolar epithelial cells (type | |
| | I and II), bronchial epithelial cells, | |
| | endothelial cells, fibroblasts | |
| | improves lung respiration | Chronic obstructive |
| | | pulmonary disorder |
| | | Reduction in protein |
| | | aggregates in cystic |
| | | fibrosis |
| | | Improved clearance of |
| | | aggresomes that |
| | | accumulate mutant cystic |
| | | fibrosis transmembrane |
| | | conductance regulator |
| | | (CFTR) protein |
| | | Improved outcome for |
| | | human idiopathic |
| | | pulmonary fibrosis |
| | dampens lung inflammation | asthma, influenza, |
| | | COPD etc. |
| Endothelial cells | Improves endothelial cell health | Improves endothelial cell |
| | | dysfunction which can |
| | | occur as a result of |
| | | diabetes, hypertension, |
| | | chronic kidney disease, |
| | | atherosclerosis and |
| | | aging |
| | Improves endothelial cell | Protects against |
| | mitochondrial function | endothelial cell injury |
| | | resulting from disease |
| | | processes such as, |
| | | diabetes, sickle cell |
| | | anaemia or thalassemia, |
| | | as well as from severe |
| | | muscle injury. |
| | | Protection of cells from |
| | | ischemia and hypoxic |
| | | conditions resulting from |
| | | myocardial function |
| | | Improved angiogenesis |
| | Improves blood flow/circulation | Peripheral artery |
| | | disease, restless leg etc. |
| | | Protection of cells during |
| | | cardiac procedures such |
| | | as angioplasty, bypass, |
| | | valve replacement |
| | Maintains endothelial cell function | |
| | Improves blood vessel health and | |
| | function | |
| Joint cartilage | Increases joint health | Reduction in |
| | | osteoarthritis in joints |
| | Enhance tendon health and/or | |
| | strength, e.g., hamstring | |
| | reduces synovitis | |
| | increases collagen production in joints | |
| | improves joint structure | |
| | reduces age-related cartilage | |
| | degeneration | |
| | Supports the health and function of | |
| | key joint cells, including chondrocytes, | |
| | synoviocytes, osteoblasts, | |
| | osteoclasts, fibroblasts, and | |
| | endothelial cells | |
| | increase cartilage cellularity | |
| Bone | Improvement of tissue mineral density | Reduces bone diseases |
| | Increased bone strength | |
| | increases bone cell differentiation | |
| Liver | Protection of liver tissue | Clearance of mutant |
| | | alpha-antitrypsin |
| | Improves liver function | Improved outcome in |
| | | non-alcoholic fatty liver |
| | | disease (NAFLD), non- |
| | | alcoholic steatohepatitis |
| | | (NASH), and alcoholic |
| | | liver disease (ALD) |
| | Improves liver metabolism | Protection against |
| | | glucose intolerance, |
| | | leptin resistance, high |
| | | cholesterol and |
| | | triglycerides in high fat |
| | | diets |
| | Improves liver detoxification | Treatment of obesity and |
| | processes | type II diabetes |
| | Increases liver ketone bodies | Improvement outcome |
| | production | from |
| | | ischemia/reperfusion |
| | | injury |
| | Supports the health and function of | Protection against drug |
| | key liver cells, including hepatocytes, | induced liver injury |
| | Kupffer cells, hepatic stellate cells, | |
| | cholangiocytes | |
| Pancreas | Improves pancreas function | Protection against |
| | | glucose intolerance, |
| | | leptin resistance, high |
| | | cholesterol and |
| | | triglycerides |
| | improves acinal cell function | Protects against diabetes |
| | | type I and II |
| | Supports the health and function of | |
| | key pancreatic cells, including insulin- | |
| | producing beta cells, alpha cells, delta | |
| | cells, pancreatic acinar cells, ductal | |
| | epithelial cells | |
| | improves glucose homeostasis | Improved outcome in |
| | | acute pancreatitis |
| Reproductive organs | Improves male fertility | Protects against PCOS |
| | Improves female fertility | Protects against |
| | | endometriosis |
| | Improves female fertility during aging | |
| | Reduces egg decline during aging | |
| | Slowing oocyte aging | |
| | improves oocyte quality | |
| | Enhancing egg fertilization | |
| | improving fertilized egg implantation | |
| | improves sperm quality | |
| | Improves sperm quantity | |
| | Improves sperm motility | |
| | increase in rapid progressive motile | |
| | sperm number | |
| | increases sperm beat cross frequency | |
| | Increasing sperm vitality | |
| | Improving sperm function | |
| | Promoting sperm maturation | |
| | Improving sperm health | |
| | Enhancing sperm mitochondrial | |
| | function | |
| | Increasing sperm mitochondrial mass | |
| | Increasing sperm mitochondrial | |
| | quantity | |
| | Enhancing oocyte mitochondrial | |
| | function | |
| | Increasing oocyte mitochondrial mass | |
| | Increasing oocyte mitochondrial | |
| | quantity | |
| | improves IVF success rate | |
| | Enhancing egg fertilization | |
| | Supports the health and function of | |
| | key reproductive organ cells, including | |
| | ovarian granulosa cells, oocytes, | |
| | Sertoli cells, Leydig cells, germ cells, | |
| | epithelial cells of the fallopian tubes | |
| | and endometrium | |
| | Supports the health and function of | |
| | key male reproductive cells, including | |
| | Sertoli cells, Leydig cells, germ cells | |
| | (spermatogonia and spermatocytes), | |
| | epididymal epithelial cells, prostate | |
| | epithelial cells | |
| | reduces symptoms of menopause | |
| Hearing organs | improves hearing | protection against |
| | | vertigo, tinnitus, |
| | | otosclerosis |
| | Supports the health and function of | |
| | key auditory cells, including cochlear | |
| | hair cells, supporting cells, spiral | |
| | ganglion neurons, strial marginal cells, | |
| | and auditory nerve fibers | |
| | reduces age-related hearing loss | |
| Kidney | improves kidney function | Podocyte resistance to |
| | | injury, which can be |
| | | caused by the following |
| | | diseases: minimal |
| | | change disease, focal |
| | | segmental |
| | | glomerulosclerosis, |
| | | diabetic nephropathy, |
| | | membrane |
| | | glomerulopathy, lupus |
| | | nephritis, and |
| | | experimental glomerular |
| | | disease |
| | Supports the health and function of | |
| | key kidney cells, including podocytes, | |
| | proximal and distal tubular epithelial | |
| | cells, collecting duct cells, mesangial | |
| | cells | |
| | improves glomerular filtration rate | protects against acute |
| | | kidney injury |
| adipose tissue | Protection against glucose | Treatment of obesity and |
| | intolerance, leptin resistance, high | type II diabetes |
| | cholesterol and triglycerides in high fat | |
| | diets | |
| | improves fatty acid oxidation | |
| | improves lipid metabolism | |

In one embodiment, the compounds of Table 1 or Table 1a enhance or maintain the health and/or function of a cell of a subject, or enhance or maintain mitophagy in a cell of a subject or enhance or maintains mitochondrial function in a cell of a subject. In an embodiment the cell is selected from the group consisting of: embryonic stem cells, induced pluripotent stem cells, adult stem cells, differentiated cells, blood cells, hematopoietic cells, epithelial cells, exocrine cells, endocrine cells, connective tissue cells, adipose cells, bone cells, smooth muscle cells, striated muscle cells, nerve cells, sensory cells, cardiac cells, hepatic cells, gastric cells, intestinal cells, pulmonary cells, kidney cells, and/or germ cells.

In a further embodiment of the invention there is provided a compound of Table 1 or Table 1a, or a salt, derivative or prodrug thereof, for use in a method of enhancing autophagy.

In a further embodiment of the invention there is provided a compound of Table 1 or Table 1a, or a salt, derivative or prodrug thereof, for use in a method of enhancing lysophagy.

In a further embodiment of the invention there is provided a compound of Table 1 or Table 1a, or a salt, derivative or prodrug thereof, for use in a method of enhancing xenophagy.

In a further embodiment of the invention there is provided a compound of Table 1 or Table 1a, or a salt, derivative or prodrug thereof, for use in a method of enhancing aggrephagy.

In a further embodiment of the invention there is provided a compound of Table 1 or Table 1a, or a salt, derivative or prodrug thereof, for use in a method of enhancing ERphagy.

In a further embodiment of the invention there is provided a compound of Table 1 or Table 1a, or a salt, derivative or prodrug thereof, for use in a method of enhancing peroxiphagy.

In a further embodiment of the invention there is provided a compound of Table 1 or Table 1a, or a salt, derivative or prodrug thereof, for use in a method of enhancing macroautophagy, microautophagy, chaperone-mediated autophagy (CMA), and/or crinophagy.

### Non-Therapeutic Uses

### Muscle Function, Performance, Endurance

Compounds of Table 1 or Table 1a, or salts, derivatives or prodrugs thereof, find use in the management of normal physiological function in healthy individuals or conditions characterised by poor physical performance, impaired endurance capacity, impaired muscle function and/or muscle wasting. Compounds of Table 1 or Table 1a, or salts, derivatives or prodrugs thereof, may improve physical performance in individuals with a disease, including young and elderly individuals. Compounds of Table 1 or Table 1a, or salts, derivatives or prodrugs thereof, may improve physical performance, for example, short-term performance or long-term performance in healthy individuals, including athletes, non-athletic individuals, sedentary individuals and the elderly. This improvement of performance may be measured by the time spent to walk or run a certain distance (for example, an improved performance during the 6-minute walk test (MWT)), an improved time to run a certain distance, an improved IPAQ score on the international physical activity questionnaire, an increased number of chair-stands in a certain time, or another test designed to measure physical performance.

Therefore, in a further embodiment of the invention, there is provided a compound of Table 1 or Table 1a, or salts, derivatives or prodrugs thereof, for improving physical performance, for improving endurance capacity and/or improving muscle function, for example, impaired muscle function.

Improved muscle function can be particularly beneficial in elderly subjects with reduced muscle function as a result of an age-related condition. For example, a subject who may benefit from improved muscle function may experience a decline in muscle function which then leads to pre-frailty and frailty. Such subjects may not necessarily experience muscle wastage in addition to their decline in muscle function. Some subjects do experience both muscle wasting and a decline in muscle function, for example subjects with sarcopenia.

In a further embodiment of the invention, there is provided a compound of Table 1 or Table 1a, or salts, derivatives or prodrugs thereof, for enhancing muscle performance. The enhanced muscle performance may be one or more improved muscle function, improved muscle strength, improved muscle endurance and improved muscle recovery.

In a further embodiment of the invention, there is provided a compounds of Table 1 or Table 1a, or salts, derivatives or prodrugs thereof, for reducing muscle wasting.

Compounds of Table 1 or Table 1a, or salts, derivatives or prodrugs thereof, further provide for the improvement of endurance capacity. The endurance capacity refers to the time to fatigue when exercising at a constant workload, generally at an intensity <80% VO₂max. Compounds of Table 1 or Table 1a, or salts, derivatives or prodrugs thereof, may improve endurance capacity in individuals with a disease, including young and elderly individuals. Compounds of Table 1 or Table 1a, or salts, derivatives or prodrugs thereof, may improve endurance capacity in healthy individuals, including athletes, non-athletic individuals, sedentary individuals and the elderly. The invention provides for a method of increasing the time to fatigue while performing a specific activity, for example, fitness training, walking, running, swimming, or cycling. This improvement of endurance capacity may be assessed with objective measurements (for example, speed, oxygen consumption or heart rate) or it can be self-reported measurements (for example, using a validated questionnaire).

Therefore, in a further embodiment of the invention, there is provided Compounds of Table 1 or Table 1a, or salts, derivatives or prodrugs thereof, for improving endurance capacity.

In a further embodiment of the invention, there is provided Compounds of Table 1 or Table 1a, or salts, derivatives or prodrugs thereof, for enhancing physical endurance in a subject, for example, in an elite athlete or sub-elite athlete. Improving physical endurance includes improving ability to perform a physical task such as exercise, physical labour, sports activities), inhibiting or retarding physical fatigue, enhancing working capacity and endurance, reducing muscle fatigue, enhancing cardiac and cardiovascular function.

In certain embodiments, the method comprises enhancing physical endurance during a high-intensity aerobic activity. In further embodiments, enhancing physical endurance comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage. In yet further embodiments, enhancing physical endurance comprises enhancing muscle endurance. In still further embodiments, enhancing physical endurance comprises increasing maximal oxygen consumption (VO2max) in the subject.

The invention further provides a compound of Table1 to improve, maintain or reduce the loss of muscle function. Compounds of Table 1 or Table 1a, or salts, derivatives or prodrugs thereof, may improve, maintain or reduce the loss of muscle function in healthy individuals, including athletes, non-athletic individuals, sedentary individuals and the elderly. For example, Compounds of Table 1 or Table 1a, or salts, derivatives or prodrugs thereof, may increase muscle strength as evidenced by the improvement of performing a physical activity, such as an exercise, for example, increased ability to lift weights or increased hand grip strength. Also, Compounds of Table 1 or Table 1a, or salts, derivatives or prodrugs thereof, may improve muscle structure, for example by increasing or maintaining muscle mass in conditions of normal muscle function, declining muscle function or impaired muscle function.

Therefore, in a further embodiment of the invention, there is provided Compounds of Table 1 or Table 1a, or salts, derivatives or prodrugs thereof, for improving muscle structure, for example by increasing or maintaining muscle mass in conditions of normal muscle function, declining muscle function or impaired muscle function.

Therefore, in a further embodiment of the invention, there is provided Compounds of Table 1 or Table 1a, or salts, derivatives or prodrugs thereof, for increasing muscle strength.

This invention further provides a composition to improve the physical performance or endurance capacity as perceived by the individual. For example, by the reduction of in perceived exertion or effort during exercise or an activity as determined using a self-reported questionnaire.

Therefore, in a further embodiment of the invention, there is provided Compounds of Table 1 or Table 1a, or salts, derivatives or prodrugs thereof, for improving physical performance and/or endurance capacity.

Muscle performance may be sports performance, which is to say the ability of an athlete's muscles to perform when participating in sports activities. Enhanced sports performance, strength, speed, and endurance are measured by an increase in muscular contraction strength, increase in amplitude of muscle contraction, or shortening of muscle reaction time between stimulation and contraction. Athlete refers to an individual who participates in sports at any level and who seeks to achieve an improved level of strength, speed, or endurance in their performance, such as, for example, body builders, bicyclists, long distance runners, and short distance runners. Enhanced sports performance is manifested by the ability to overcome muscle fatigue, reduced muscle fatigue, ability to maintain activity for longer periods of time, and have a more effective workout.

Therefore, in a further embodiment of the invention, there is provided Compounds of Table 1 or Table 1a, or salts, derivatives or prodrugs thereof, for enhancing sports performance.

According to a further embodiment of the invention, there is provided Compounds of Table 1 or Table 1a, or salts, derivatives or prodrugs thereof, for use in a method of enhancing physical performance in a subject, for example, in an elite athlete or sub-elite athlete.

In certain embodiments, enhancing physical performance comprises at least one effect selected from the group consisting of enhancing athletic performance, enhancing running performance, enhancing muscle performance, enhancing aerobic endurance, enhancing the rating of or lowering perceived exertion, lowering post exercise fatigue, enhancing muscle recovery, reducing exercise-induced muscle damage, reducing muscle soreness, and enhancing repair of exercise-induced muscle damage. In further embodiments, enhancing physical performance comprises enhancing muscle performance during a high-intensity aerobic activity. In yet further embodiments, enhancing physical performance comprises increasing aerobic endurance during a high-intensity aerobic activity. In still further embodiments, enhancing physical performance comprises increasing resting metabolic rate (RMR). In certain embodiments, enhancing physical performance results in an improvement in athletic performance. In further embodiments, enhancing physical performance results in an improvement in footrace completion times. In yet further embodiments, enhancing physical performance results in a decrease in Ratings of Perceived Exertion (RPE).

According to a further embodiment of the invention, there is provided a compound of Table 1 or Table 1a, or salts, derivatives or prodrugs thereof for use in a method of enhancing physical recovery in a subject, for example, in an elite athlete or sub-elite athlete.

In certain embodiments, physical recovery is enhanced after a high-intensity aerobic activity. In further embodiments, enhancing physical recovery comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage. In yet further embodiments, enhancing physical recovery comprises enhancing muscle recovery after a high-intensity aerobic activity. In still further embodiments, enhancing physical recovery comprises reducing muscle soreness after a high-intensity aerobic activity. In certain embodiments, enhancing physical recovery comprises lowering creatine kinase (CK) levels in the subject, compared to baseline, following an aerobic activity as measured by area under the plasma concentration-time curve of CK (AUCCK). In further embodiments, enhancing recovery comprises lowering C-reactive protein (CRP) levels in the subject, compared to baseline, following an aerobic activity as measured by area under the plasma concentration-time curve (AUCCRP).

### Immune Health

In a further embodiment of the invention there is provided the use of a compound of Table 1 or Table 1a, or salts, derivatives or prodrugs thereof, for maintaining or enhancing immune health.

In a further embodiment of the invention there is provided the use of a compound of Table 1 or Table 1a, or salts, derivatives or prodrugs thereof, for reducing or slowing inflammaging.

In a further embodiment of the invention there is provided the use of a compound of Table 1 or Table 1a, or salts, derivatives or prodrugs thereof, for slowing immune aging.

In a further embodiment of the invention there is provided the use of a compound of Table 1 or Table 1a, or salts, derivatives or prodrugs thereof, for reducing immune cell aging.

In a further embodiment of the invention there is provided the use of a compound of Table 1 or Table 1a, or salts, derivatives or prodrugs thereof, for boosting immune function.

In a further embodiment of the invention there is provided the use of a compound of Table 1 or Table 1a, or salts, derivatives or prodrugs thereof, for supporting a healthy immune system.

In a further embodiment of the invention there is provided the use of a compound of Table 1 or Table 1a, or salts, derivatives or prodrugs thereof, for prevention or management of or reduction of the frequency of colds and/or flu.

In a further embodiment of the invention there is provided the use of a compound of Table 1 or Table 1a, or salts, derivatives or prodrugs thereof, for the treatment of or prevention of infection.

In a further embodiment of the invention there is provided the use of a compound of Table 1 or Table 1a, or salts, derivatives or prodrugs thereof, for improving vaccination response.

In a further embodiment of the invention there is provided the use of a compound of Table 1 or Table 1a, or salts, derivatives or prodrugs thereof, for enhancing an immune response against infection.

In a further embodiment of the invention there is provided the use of a compound of Table 1 or Table 1a, or salts, derivatives or prodrugs thereof, for protecting against infection, for example, protection against colds and flu.

In a further embodiment of the invention there is provided the use of a compound of Table 1 or Table 1a, or salts, derivatives or prodrugs thereof, for immune support.

In a further embodiment of the invention there is provided the use of a compound of Table 1 or Table 1a, or salts, derivatives or prodrugs thereof, for reducing the risk of respiratory tract infections (for example, colds, flu or pneumonia).

In a further embodiment of the invention there is provided the use of a compound of Table 1 or Table 1a, or salts, derivatives or prodrugs thereof, for increasing the rate of recovery from respiratory tract infections (for example, colds, flu or pneumonia).

In a further embodiment of the invention there is provided the use of a compound of Table 1 or Table 1a, or salts, derivatives or prodrugs thereof, for increasing the quantity of CD8 positive T-cells, for example, naïve CD8-positive T-cells.

In a further embodiment of the invention there is provided the use of a compound of Table 1 or Table 1a, or salts, derivatives or prodrugs thereof, for increasing the performance or activity of CD8 positive T-cells, for example, naïve CD8-positive T-cells.

In a further embodiment of the invention there is provided the use of a compound of Table 1 or Table 1a, or salts, derivatives or prodrugs thereof, for use in a method of slowing immune aging by enhancing naïve T cells.

### Brain Health

According to a further embodiment of the invention there is provided the use of a compound of Table 1 or Table 1a, or salts, derivatives or prodrugs thereof, for maintaining or enhancing brain health.

According to a further embodiment of the invention there is provided the use of a compound of Table 1 or Table 1a, or salts, derivatives or prodrugs thereof, for promoting healthy brain function.

According to a further embodiment, there is provided a compound of Table 1 or Table 1a, or salts, derivatives or prodrugs thereof, for use for optimising brain health.

According to a further embodiment, there is provided a compound of Table 1 or Table 1a, or salts, derivatives or prodrugs thereof, for use to improve, protect, and maintain brain function and cognition.

According to a further embodiment, there is provided a compound of Table 1 or Table 1a, or salts, derivatives or prodrugs thereof, for use for enhancing mental alertness, for example, mental accuracy and clearer/sharper thinking.

According to a further embodiment, there is provided a compound of Table 1 or Table 1a, or salts, derivatives or prodrugs thereof, for use for increasing psychomotor speed and/or reaction time.

According to a further embodiment, there is provided a compound of Table 1 or Table 1a, or salts, derivatives or prodrugs thereof, for use for enhancing/improving memory.

According to a further embodiment, there is provided a compound of Table 1 or Table 1a, or salts, derivatives or prodrugs thereof, for use for enhancing/improving learning.

According to a further embodiment, there is provided a compound of Table 1 or Table 1a, or salts, derivatives or prodrugs thereof, for use for enhancing/improving reasoning, for example, promoting mental processing.

According to a further embodiment, there is provided a compound of Table 1 or Table 1a, or salts, derivatives or prodrugs thereof, for use for enhancing/improving focus, for example, concentration, complex attention and/or sustained attention.

According to a further embodiment, there is provided the use of a compound of Table 1 or Table 1a, or salts, derivatives or prodrugs thereof, for stress relief and/or anxiety relief.

According to a further embodiment, there is provided the use of a compound of Table 1 or Table 1a, or salts, derivatives or prodrugs thereof, for treating stress and/or anxiety.

According to a further embodiment, there is provided the use of a compound of Table 1 or Table 1a, or salts, derivatives or prodrugs thereof, for treating depression.

According to a further embodiment, there is provided the use of a compound of Table 1 or Table 1a, or salts, derivatives or prodrugs thereof, for improving mood.

According to a further embodiment, there is provided the use of a compound of Table 1 or Table 1a, or salts, derivatives or prodrugs thereof, for enhancing sleep and/or relaxation.

### Skin

According to a further embodiment, there is provided the use of a compound of Table 1 or Table 1a, or salts, derivatives or prodrugs thereof, for treating or preventing skin conditions in generally healthy subjects. For example, it is suitable for use by subjects with skin prone to the ailments mentioned herein, for example subjects with intermittent skin issues, for example, skin prone to sunburn, prone to acne, prone to inflammation and prone to psoriasis. According to a further embodiment, there is provided the use of a compound of Table 1 or Table 1a, or salts, derivatives or prodrugs thereof, for treating or preventing skin conditions in generally healthy subjects with the proviso that the following compounds are excluded: chrysoeriol and juglanin and n-norluciferine.

According to a further embodiment of the invention, there is provided use of a compound of Table 1 or Table 1a, or salts, derivatives or prodrugs thereof, for one or more of the following:
a) maintaining or enhancing skin health and appearance;
b) for maintaining or enhancing skin energy;
c) for maintaining or enhancing skin collagen;
d) for maintaining or enhancing skin elasticity
e) reducing skin biological aging;
f) supporting healthy skin aging;
g) reducing skin wrinkles, and/or fine lines;
h) improving skin mitochondrial function;
i) maintaining or improving skin hydration;
j) promoting replenishment of skin ceramide levels;
k) improving barrier function;
l) protecting against free radical damage; and/or
m) improving skin dryness;
n) improving photodamaged skin;
o) Improving skin health in skin prone to redness, inflammation, acne or dryness
p) helping replenish ceramide stores; and
q) reducing skin redness and irritation.

In a further embodiment of the invention, there is provided a compound of Table 1 or Table 1a, or salts, derivatives or prodrugs thereof, for one or more of the following uses:
(a) skin bleaching and/or lightening skin colour and/or lightening skin tone;
(b) skin whitening;
(c) protection of skin caused by damage by the environment (For example, damage caused by sunlight/UV irradiation and/or other sources of irradiation (α, β and γ radiation) and/or damage caused by pollution);
(d) decreasing pigmentation; and
(e) suppressing melanin production.

In a further embodiment of the invention, there is provided a compound of Table 1 or Table 1a, or salts, derivatives or prodrugs thereof, for one or more of the following uses:
(a) renewal and/or revitalization of skin appearance;
(b) enhancement of skin energy supply;
(c) improvement in skin texture;
(d) enhancement of skin radiance;
(e) skin rejuvenation/regeneration;
(f) reduction of pore and micro line visibility;
(g) improvement in skin respiration.
(h) support optimal cellular function in skin;
(i) promote/activate skin detoxifying processes;
(j) promote collagen repair, and/or
(k) improve skin stem cell health.

In a further embodiment of the invention, there is provided a compound of Table 1 or Table 1a, or salts, derivatives or prodrugs thereof, for one or more of the following uses:
(a). Treating or preventing skin inflammation, for example, inflammation caused by radiation, for example, UV radiation or collagen degrading enzymes;
(b). maintaining or enhancing skin health and/or appearance;
(c). maintaining or enhancing skin cell mitochondrial function;
(d). reducing or reversing skin aging;
(e). enhancing skin longevity;
(f). reducing or preventing skin wrinkles; and/or
(g). enhancing skin healthspan;

### Hair

According to a further aspect of the invention there is provided a compound of Table 1 or Table 1a, or salt, derivative or prodrug thereof, for stimulating hair growth.

According to a further aspect of the invention there is provided a compound of Table 1 or Table 1a, or salt, derivative or prodrug thereof for the treatment of hair loss

According to a further aspect of the invention there is provided a compound of Table 1 or Table 1a, or salt, derivative or prodrug thereof, for preventing or ameliorating hair loss.

According to a further aspect of the invention there is provided a compound of Table 1 or Table 1a, or salt, derivative or prodrug thereof, for preventing or reducing hair loss.

According to a further aspect of the invention there is provided a compound of Table 1 or Table 1a, or salt, derivative or prodrug thereof, for preventing or ameliorating hair loss in a subject prone to hair loss, for example a male or female subject. Such hair loss may be caused by a number of factors, for example, heredity, hormonal changes, physical or emotional stress, medication and/or poor nutrition.

According to a further aspect of the invention there is a compound of Table 1 or Table 1a, or salt, derivative or prodrug thereof, for delaying the onset of hair loss.

According to a further aspect of the invention there is provided a compound of Table 1 or Table 1a, or salt, derivative or prodrug thereof, for treating hair thinning.

According to a further aspect of the invention there is provided a compound of Table 1 or Table 1a, or salt, derivative or prodrug thereof, for use in a method of increasing hair thickness (i.e. number of hair fibres per surface area) and/or number hair follicles actively producing hair fibres.

According to a further aspect of the invention there is provided a compound of Table 1 or Table 1a, or salt, derivative or prodrug thereof, for slowing or preventing premature greying of hair.

In a further embodiment of the invention, there is provided a compound of Table 1 or Table 1a, or salt, derivative or prodrug thereof, for one or more of the following uses:
(a) maintaining or enhancing hair thickness;
(b) hair follicle cell regeneration;
(c) hair follicle cell survival;
(d) hair stem cell growth and/or regeneration;
(e) hair cell survival;
(f) hair loss prevention,
(g) promoting new hair growth;
(h) hair maintenance;
(i) scalp health improvement,
(j) improving or maintaining hair strength,
(k) or improving survival of hair transplants; and/or
(l) hair growth restoration promotion.

In a further embodiment of the invention, there is provided a compound of Table 1 or Table 1a, or salt, derivative or prodrug thereof, for one or more of the following uses:
a) enhancing hair stem cell function;
b) enhancing hair follicle elongation,
c) enhancing hair matrix proliferation, for example, as measured by Ki67 levels;
d) enhancing hair growing phase, (enhancing the duration of the anaphase);
e) hair matrix proliferation,
f) inhibition of hair matrix apoptosis; and
g) reducing melanin clumping, for example, in pigmentary units, for example, in hair follicle pigmentary units.

In a further embodiment of the invention, there is provided a compound of Table 1 or Table 1a, or salt, derivative or prodrug thereof, for one or more of the following uses:
(a) maintaining or enhancing hair health and/or appearance;
(b). treating hair loss;
(c). delaying the onset of hair loss;
(d). stimulating hair growth;
(e). increasing hair thickness;
(f). improving or maintaining hair strength; and/or
(g). slowing or preventing premature greying of hair.

### Nails

Compounds of Table 1 or Table 1a, or salts, derivatives or prodrugs thereof find use in the treatment or prevention of diseases, disorders and conditions associated with nails. Such uses in diseases, disorders and conditions include use in both pathological and non-pathological conditions and cosmetic indications.

In a further embodiment of the invention, nail diseases, disorders and conditions include nail deformities (changes in nail shape) and nail dystrophies (changes in nail texture, colour or both.

Nail conditions include: brittle nails, triangular worn down nails, trachyonychia, and habit tic deformity.

Therefore, according to a further embodiment of the invention, there is provided a compound of Table 1 or Table 1a, or salt, derivative or prodrug thereof, for use in the treatment or prevention of diseases, disorders and conditions associated with nails, for example, nail deformities (changes in nail shape) and nail dystrophies (changes in nail texture, colour or both and brittle nails, triangular worn down nails, trachyonychia, and habit tic deformity.

### Inflammation

According to a further aspect of the invention there is provided a compound of Table 1 or Table 1a, or salt, derivative or prodrug thereof, for the treatment or prevention of inflammation in generally healthy individuals.

According to a further aspect of the invention there is provided a compound of Table 1 or Table 1a, or salt, derivative or prodrug thereof, for slowing or preventing inflammaging.

### Oral Care

In a further embodiment, there is provided a non-therapeutic use for treating a condition comprising administration of a composition of the invention wherein the use is selected from one or more of the following:
(a). enhancing oral healthspan;
(b). enhancing oral longevity;
(c). enhancing gum healthspan;
(d). enhancing tongue healthspan;
(e). enhancing tongue longevity;
(f). maintaining or enhancing oral muscosa health;
(g). maintaining or enhancing oral epithelium health;
(h). maintaining or enhancing gum health;
(i). reducing gum sensitivity;
(j). treating, managing or preventing gingivitis;
(k). treating, managing or preventing periodontitis (gum disease;
(l). treating, managing or preventing inflammation in the oral cavity, for example, gum inflammation;
(m). treating, managing or preventing receding gums; and/or
(n). reducing teeth hypersensitivity
(o). reducing periodontitis induced bone loss.
(p). reducing gum inflammation and gingival inflammation
(q). Improving gingival cell function, including gingival fibroblasts
(r). Improving function of the tongue, including stratified squamous epithelial keratinocytes, basal epithelial stem/progenitor cells, keratinized epithelial cells (filiform papillae), taste bud Type I (supporting) cells, taste bud Type II (sweet/bitter/umami receptor) cells, taste bud Type III (presynaptic/sour) cells, taste bud Type IV (basal progenitor) cells, primary sensory neurons (chorda tympani, glossopharyngeal, trigeminal afferents), free nerve endings, serous salivary acinar cells, mucous salivary acinar cells, salivary ductal epithelial cells, myoepithelial cells, Langerhans cells, macrophages, T lymphocytes (including tissue-resident memory T cells), neutrophils, fibroblasts, endothelial cells, lymphatic endothelial cells, skeletal muscle myocytes

In a further embodiment, there is provided a non-therapeutic use for a condition, wherein the condition is selected from one or more of the following:
(a). reduction of hypersensitivity of the teeth;
(b). reduction of bad breadth (halitosis);
(c). reduction of plaque accumulation;
(d). reduction of tartar build-up;
(e). treatment, relieving or reduction of dry mouth;
(f). cleaning the teeth and/or oral cavity; and/or
(g). maintaining or enhancing the mouth microbiome; and/or
(h). prevention of stains and/or for whitening teeth;
comprising the administration of a composition of the invention, for example, administration to the oral cavity.

In a further embodiment there is provided use of a composition of the invention for use in subjects with sensitive gums. In a further embodiment, the is provided use of a composition for reducing gum sensitivity.

In a further embodiment, there is provided a composition of the invention for maintaining healthy gums, for the treatment of irritated gums, and for the treatment of patients prone to gingivitis.

In a further embodiment, there is provided a composition of the invention for maintaining or enhancing gum health.

In a further embodiment, there is provided a composition of the invention for reducing or preventing gum bleeding.

In a further embodiment, there is provided a composition of the invention for preventing or reducing gum inflammation.

In a further embodiment, there is provided a composition of the invention for treating irritated gums.

In a further embodiment, there is provided the use of a composition of the invention in a method for enhancing gum longevity.

In a further embodiment, there is provided the use of a composition of the invention in a method for enhancing gum healthspan.

In a further embodiment, there is provided the use of a composition of the invention in a method of facilitating gum regeneration.

In a further embodiment, there is provided the use of a composition of the invention in a method or enhancing or maintaining oral bone health.

In a further embodiment, there is provided the use of a composition of the invention in a method or enhancing or maintaining oral bone health during aging.

In a further embodiment, there is provided the use of a composition of the invention in a method of maintaining or enhancing oral bone density.

In a further embodiment, there is provided the use of a composition of the invention in a method of maintaining or enhancing oral bone density during aging.

In a further embodiment, there is provided the use of a composition of the invention in a method or reducing decreases oral bone health due to aging.

### Therapeutic Uses

### Inflammatory Diseases, Disorders and Conditions

According to one embodiment of the invention, there is provided a compound of Table 1 or Table 1a, or salt, derivative or prodrug thereof, for use in relieving or treating acute inflammation or chronic inflammation-induced diseases. In one embodiment, the acute inflammation is, for example, influenza virus pneumonia, new coronavirus pneumonia, and acute lung injury inflammation. In a further embodiment, the chronic inflammation is, for example, chronic obstructive pneumonia, chronic asthmatic airway inflammation and chronic obstructive pulmonary disease (COPD).

According to a further embodiment of the invention, there is provided a compound of Table 1 or Table 1a, or salts, derivatives or prodrugs thereof, for treating or preventing an inflammatory disease, for example, an inflammatory disease selected from inflammatory skin diseases, Crohn's disease, ulcerative colitis, peritonitis, osteomyelitis, meningitis, meningitis, encephalitis, pancreatitis, trauma-induced shock, bronchial asthma, allergic rhinitis, cystic fibrosis, Inflammatory bowel disease, acute bronchitis, chronic bronchitis, osteoarthritis, gout, spondyloarthropathies, ankylosing spondylitis, intestinal spondylitis, inflammatory arthropathy, ankylosing spondylitis, reactive arthropathy, infectious arthritis, systemic lupus erythematosus, recurrent, and psoriasis. Any one selected from arthritis, gonococcal arthritis, tuberculous arthritis, viral arthritis, fungal arthritis, rheumatoid arthritis, rheumatoid polyposis muscle pain, arthritic arthritis, calcium arthritis, non-articular rheumatism, bursitis, hay fever.

### Infectious Diseases

According to one embodiment of the invention, there is provided a compound of Table 1 or Table 1a, or salts, derivatives or prodrugs thereof, for use in the treatment of microbial diseases. In a further embodiment, there is provided a compound of Table 1 or Table 1a, or salts, derivatives or prodrugs thereof, for use in the treatment of viral diseases.

According to a future embodiment of the invention, there is provided a compound of Table 1 or Table 1a, or salts, derivatives or prodrugs thereof, for use in treating a post-viral illness, for example, wherein the compound treats symptoms associated with recovery from the viral disease or infection in the subject. Symptoms associated with recovery from a viral disease comprise fatigue, post-exertional malaise (PEM), problems with memory or concentration, sore throat, headache, muscle or joint pain, dizziness, brain fog, shortness of breath, and/or unrefreshing sleep. In one embodiment, the post-viral illness is selected from myalgic encephalomyelitis/chronic fatigue syndrome, long-COVID (for example, caused by an infection with a delta variant or an omicron variant of SARS-COV-2 i) or chronic fatigue syndrome.

### Immune Function

In a further embodiment of the invention there is provided the use of a compound of Table 1 or Table 1a, or salt, derivative or prodrug thereof, for supporting and/or enhancing immune function.

According to a further aspect of the invention there is provided a compound of Table 1 or Table 1a, or salt, derivative or prodrug thereof, for use in a method of (i) raising an immune response to an antigen and/or (ii) enhancing, modulating or augmenting an immune response to an antigen in a human or animal subject.

According to a further aspect of the invention there is provided a compound of Table 1 or Table 1a, or salt, derivative or prodrug thereof, for use in a method of preventing, reducing or slowing inflamm-ageing or inflammaging in a human or animal subject.

In a further embodiment of the invention there is provided the use of a compound of Table 1 or Table 1a, or salt, derivative or prodrug thereof, for use as an anti-inflammatory agent.

In a further embodiment of the invention there is provided the use of a compound of Table 1 or Table 1a, or salt, derivative or prodrug thereof, for use for the prevention, treatment, and management of a condition selected from acne, eczema, psoriasis and rosacea.

According to a further aspect of the invention there is provided a compound of Table 1 or Table 1a, or salt, derivative or prodrug thereof, for use in a method of treating immune-senescence.

According to a further aspect of the invention there is provided a compound of Table 1 or Table 1a, or salt, derivative or prodrug thereof, for use in a method of preventing, reducing or slowing stem cell senescence in a human or animal subject.

In a further embodiment of the invention there is provided the use of a compound of Table 1 or Table 1a, or salt, derivative or prodrug thereof, for supporting and/or enhancing immune function during or after cancer treatment.

In a further embodiment of the invention there is provided the use of a compound of Table 1 or Table 1a, or salt, derivative or prodrug thereof, for supporting and/or enhancing immune function remission after cancer treatment.

In a further embodiment of the invention there is provided the use of a compound of Table 1 or Table 1a, or salt, derivative or prodrug thereof, or supporting and/or enhancing immune function during and after hospital admissions.

### Cardiovascular and Cerebrovascular Diseases

According to one embodiment of the invention, there is provided a compound of Table 1 or Table 1a, or salts, derivatives or prodrugs thereof, for use in the treatment of cardiovascular and cerebrovascular diseases. For example, cardiovascular and cerebrovascular diseases selected from transient ischemia, hypertension, anti-platelet aggregation, formation of the internal carotid artery onset plate, Cerebral atherosclerosis, arterial atherosclerosis, infarction, myocarditis, meningitis, cerebral arteriosclerosis, hyperlipidaemia and basilar artery insufficiency. In a further embodiment, the cardiovascular and cerebrovascular diseases are selected from cerebral ischemia, cerebral thrombosis, cerebral embolism, cerebral stroke, hypertension and coronary heart disease, myocardial ischemia, arrhythmia, heart failure and angina pectoris.

### Metabolic Diseases

According to one embodiment of the invention, there is provided a compound of Table 1 or Table 1a, or salts, derivatives or prodrugs thereof, for use in the treatment of metabolic diseases. For example, wherein the metabolic diseases are selected from: diabetes mellitus, obesity, metabolic syndrome, reduced metabolic rate, fatty liver disease (for example NAFLD and NASH), and decline in liver function. In a further embodiment a compound of Table 1 or Table 1a, or salts, derivatives or prodrugs thereof, are useful for weight management.

### Neurological Disorders

According to one embodiment of the invention, there is provided a compound of Table 1 or Table 1a, or salts, derivatives or prodrugs thereof, for use in the treatment of neurological disorders, including neurodegenerative disorders. Examples of neurological disorders include: cognitive decline, memory decline, anxiety, depression, epilepsy, stroke, amyotrophic lateral sclerosis, dementia (e.g. vascular dementia, Lewy Body Dementia, Frontotemporal Dementia and Alzheimer's disease), Parkinson's disease, and Huntington disease.

According to one embodiment of the invention, there is provided a compound of Table 1 or Table 1a, or salts, derivatives or prodrugs thereof, for use in the treatment of neurological disorders, wherein the neurological disorders are selected from early on-set dementia and/or mild cognitive impairment,

According to a further embodiment of the invention, there is provided a compound of Table 1 or Table 1a, or salts, derivatives or prodrugs thereof, for use in the prophylaxis or treatment of a disease state initiated or characterized (i) by a decline in cognitive function; or (ii) by mood disturbances. Such disease states can include, without limitation, neurodegenerative disease, cognitive disorder, mood disorder and stress and/or anxiety disorder.

An embodiment of the invention is a method of improving cognitive function. The method includes the step of administering to a subject in need thereof an effective amount of a compound of Table 1 or Table 1a, or salts, derivatives or prodrugs thereof, to improve cognitive function. In one embodiment, the cognitive function is selected from the group consisting of perception, memory, attention, speech comprehension, speech generation, reading comprehension, creation of imagery, learning, and reasoning. In one embodiment, the cognitive function is selected from the group consisting of perception, memory, attention, and reasoning. In one embodiment, the cognitive function is memory.

According to a further embodiment, there is provided a compound of Table 1 or Table 1a, or salts, derivatives or prodrugs thereof, for use to improve, protect, and maintain brain function and cognition.

According to a further embodiment, there is provided a compound of Table 1 or Table 1a, or salts, derivatives or prodrugs thereof, for use protecting against brain oxidative stress and/or reducing brain inflammation

### Cancer

According to one embodiment of the invention, there is provided a compound of Table 1 or Table 1a, or salts, derivatives or prodrugs thereof, for use in the treatment of cancer. Examples of suitable cancers include: bladder cancer, B-cell lymphoma such as Hodgkin's lymphoma, T-cell lymphoma, T-cell acute lymphoblastic leukemia, acute lymphoblastic leukemia, chrome lymphoblastic leukemia, acute myelogenous leukemia, chronic myelogenous leukemia, erythroleukemia, triple negative breast cancer, breast cancer, ovarian cancer, melanoma including paediatric melanoma, lung cancer such as squamous cell lung carcinoma and non small-cell lung cancer, pancreatic cancer, glioblastoma, colorectal cancer, head and neck cancer such a head and neck squamous cell carcinoma, cervical cancer, prostate cancer, liver cancer, oral squamous cell carcinoma, skin cancer, medulloblastoma, hepatocellular carcinoma, intrahepatic and extrahepatic cholangiocarcinoma, desmoid tumours, soft tissue sarcoma, adenoid cystic carcinoma, urothelial cancer, renal cancer, hepatocellular cancer, skin cancer, such as Merkel cell carcinoma, gastric cancer and gastroesophageal cancer.

According to a further embodiment of the invention, there is provided a compound of Table 1 or Table 1a, or salts, derivatives or prodrugs thereof, for inhibiting metastasis.

### Muscle Pathological Conditions

Compositions of the invention find use in the treatment of muscle-related pathological conditions. Therefore, according to a further embodiment of the invention, there is provided a compound of Table 1 or Table 1a, or salts, derivatives or prodrugs thereof, for use in the treatment or prevention of muscle pathological conditions. Muscle-related conditions include both conditions impacting generally healthy individuals as well as pathological conditions. Such muscle conditions found in healthy people or people affected by a disease include musculoskeletal diseases or disorders; cachexia; muscle wasting; age related decline in muscle function; pre-frailty; frailty; myopathies; neuromuscular diseases, such as Duchenne muscular dystrophy and other dystrophies; age-related sarcopenia; acute sarcopenia; muscle atrophy and/or cachexia, for example muscle atrophy and/or cachexia associated with burns, bed rest, limb immobilization, or major surgery, including thoracic, abdominal, and/or orthopaedic surgery; and muscle degenerative disease.

Examples of age-related conditions that may be treated with compositions of the invention include sarcopenia and muscle wasting.

The myopathy may also be caused by a muscular dystrophy syndrome, such as Duchenne.

Compounds of Table 1 or Table 1a, or salts, derivatives or prodrugs thereof, may improve, maintain or reduce the loss of muscle function in individuals with a disease, including young and elderly individuals.

### Skin

According to a further aspect of the invention, there is provided a compound of Table 1 or Table 1a, or salts, derivatives or prodrugs thereof, for the treatment of a skin condition, disease or disorder. Examples of skin diseases, disorders or conditions are selected from the group consisting of melasma, chloasma, hyperpigmentation, skin-aging, liver spots, lentigo, inflammation of the skin, skin irritation, skin infection, warts, psoriasis, and protection of skin from damage caused by the environment and/or therapy. The skin disease, disorder or condition is also selected from melanosis, dermatitis, linea nigra and endocrine diseases such as Addison's and Cushing's syndrome. A compound of Table 1 or Table 1a, or salts, derivatives or prodrugs thereof, for the treatment of a skin condition, disease or disorder with the proviso that the compounds chrysoeriol, and juglanin are excluded.

In a further embodiment of the invention, skin diseases, disorders and conditions include:
(a) skin aging,
(b) age spots;
(c) liver spots;
(d) dry skin;
(e) radiation induced skin damage (for example, IR, UV, alpha particles, beta particles of gamma irradiation);
(f) lentigo;
(g) hyperpigmentation, for example age-related hyperpigmentation of the skin, or post-inflammatory hyperpigmentation;
(h) melasma (chloasma/mask of pregnancy);
(i) skin irritation, for example, dermatitis;
(j) skin infection, for example, warts;
(k) inflammatory skin conditions (for example, atopic eczema, seborrhoeic eczema, polymorphous photodermatosis, psoriasis, vitiligo),
(l) uneven skin colour (smoothing out thereof),
(m) fine lines and/or wrinkles;
(n) linea nigra;
(o) melanosis;
(p) endocrine diseases, such as Addison's and Cushing's syndrome;
(q) stretch marks of the skin;
(r) skin inflammation, including irritation, sensitivity and itching.

In a further embodiment of the invention, there is provided a compound of Table 1 or Table 1a, derivatives or prodrugs thereof, for maintaining or enhancing keratinocyte health and/or function.

In a further embodiment of the invention, there is provided a compound of Table 1 or Table 1a, or salts, derivatives or prodrugs thereof, for enhancing keratinocyte differentiation. In a further embodiment of the invention, there is provided a compound of Table 1 or Table 1a, or salts, derivatives or prodrugs thereof, for enhancing or maintaining keratinocyte skin barrier function.

In a further embodiment of the invention, there is provided a compound of Table 1 or Table 1a, or salts, derivatives or prodrugs thereof, for enhancing or maintaining keratinocyte wound healing function.

In a further embodiment of the invention, there is provided a compound of Table 1 or Table 1a, or salts, derivatives or prodrugs thereof, for enhancing or maintaining keratinocyte skin repair function.

In a further embodiment of the invention, there is provided a compound of Table 1 or Table 1a, or salts, derivatives or prodrugs thereof, for enhancing or maintaining keratinocyte immune function.

In a further embodiment of the invention, there is provided a compound of Table 1 or Table 1a, or salts, derivatives or prodrugs thereof, for enhancing or maintaining keratinocyte skin remodelling function.

In a further embodiment of the invention, there is provided a compound of Table 1 or Table 1a, or salts, derivatives or prodrugs thereof, for increasing keratinocyte proliferation.

In a further embodiment of the invention, there is provided a compound of Table 1 or Table 1a, or salts, derivatives or prodrugs thereof, for maintaining or enhancing skin fibroblast health and/or function.

In a further embodiment of the invention, there is provided a compound of Table 1 or Table 1a, or salts, derivatives or prodrugs thereof, for increasing skin fibroblast proliferation.

In a further embodiment of the invention, there is provided a compound of Table 1 or Table 1a, or salts, derivatives or prodrugs thereof, for increasing skin fibroblast wound healing function.

In a further embodiment of the invention, there is provided a compound of Table 1 or Table 1a, or salts, derivatives or prodrugs thereof, for increasing skin fibroblast skin repair function.

In a further embodiment of the invention, there is provided a compound of Table 1 or Table 1a, or salts, derivatives or prodrugs thereof, for increasing skin fibroblast skin remodelling function

### Hair

In further embodiments of the invention, there is provided a compound of Table 1 or Table 1a, or salt, derivative or prodrug thereof, for use for treating or preventing hair diseases disorders or conditions.

In one embodiment, hair diseases, disorders and conditions are selected from denutrition-based alopecia, endocrine disorder-based alopecia, vascular disorder-based alopecia, alopecia premature, traction alopecia, alopecia areata, alopecia neurotica, pityriasis alopecia, Trichotillomania, alopecia maligna, female pattern alopecia, male pattern alopecia, androgenetic alopecia, telogen effluvium, tinea capitis, alopecia totalis hypotrichosis, genetic hypotrichosis simplex, systemic drug for alopecia-based hair-loss, mechanical hair-loss, traumatic alopecia, pressure alopecia, anagen effluvium, pityriasis alopecia, alopecia syphilltica, lopecia seborrheica, symptomatic alopecia, alopecia cicatrisata, and alopecia congenita. However, the hair-loss should be understood as meaning including all symptoms classified as the alopecia in this field, regardless of the direct or indirect cause of the occurrence of the hair-loss.

According to a further aspect of the invention there is provided a compound of Table 1 or Table 1a, or salt, derivative or prodrug thereof, for the treatment of hair loss, wherein the hair loss is caused by one or more of the following: skin disorders, a medicine, a disease, autoimmunity, iron deficiency, severe stress, scalp radiation, pregnancy or pulling at your own hair.

### Aging/Healthspan

According to a further embodiment of the invention, there is provided a compound of Table 1 or Table 1a, or salts, derivatives or prodrugs thereof, for use in the increasing or prolonging of healthspan. Healthspan can be defined as the part of a person's life during which they are generally in good health.

In a further embodiment of the invention, there is provided a compound of Table 1 or Table 1a, or salt, derivative or prodrug thereof, for slowing aging.

In a further embodiment of the invention, there is provided a compound of Table 1 or Table 1a, or salt, derivative or prodrug thereof, for promoting healthy aging.

In further embodiments of the invention, there is provided a compound of Table 1 or Table 1a, or salt, derivative or prodrug thereof for use for reducing the impact of chronological aging on biological aging.

### Fertility

According to a further embodiment of the invention, there is provided a compound of Table 1 or Table 1a, or salts, derivatives or prodrugs thereof, for use for one or more of the following:
a) enhancing or improving female fertility;
b) improving probability of oocyte fertilization;
c) enhancing the probability of implantation;
d) improving the probability of pregnancy
e) increasing the quality or quantity of eggs;
f) slowing oocyte aging;
g) restoring oocyte maturation;
h) maintaining or enhancing ovarian health;
i) maintaining or enhancing ovarian reserve in a female subject;
j) increasing in oocyte mitochondrial mass;
k) preventing a decrease in oocyte mitochondrial mass;
l) enhancing embryo development

According to a further embodiment of the invention, there is provided a compound of Table 1 or Table 1a, or salts, derivatives or prodrugs thereof, for use for one or more of the following:
a) enhancing or improving male fertility;
b) improving sperm quality;
c) increasing sperm quantity;
d) improving sperm motility;
e) improving sperm maturation;
f) improving sperm vitality, and/or
g) improving sperm function.

According to a further embodiment of the invention, there is provided a compound of Table 1 or Table 1a, or salts, derivatives or prodrugs thereof, the enhancement or improvement in fertility in a male subject, comprises, one of more of the following:
a) an improvement in sperm quality;
b) an increase in sperm quantity;
c) an improvement in sperm motility;
d) an increase in rapid progressive motile sperm quantity;
e) an increase in sperm beat cross frequency;
f) an improvement in sperm maturation;
g) an improvement in sperm vitality,
h) an improvement in sperm function; and/or.
i) An improvement in sperm health

In a further embodiment, the enhancement or improvement in fertility comprises an increase in sperm quality and/or sperm motility.

In a further embodiment, the enhancement or improvement in fertility is in a male subject, and comprises, one of more of the following:
(a) An enhancement of sperm mitochondrial function;
(b) An increase in sperm mitochondrial mass, and or
(c) An increase in the quantity of mitochondria in sperm.

### Treatment of Oral Diseases and Disorders

In a further embodiment of the invention, there is provided a composition of the invention for use in a disease disorder or condition, selected from one or more of the following:
(a). treatment or prevention of erosive tooth demineralization;
(b). reduction or inhibition formation of dental caries;
(c). reduction, repair or inhibition of pre-carious lesions of the enamel, e.g., as detected by quantitative light-induced fluorescence (QLF) or electrical caries measurement (ECM);
(d). reduction or inhibition of demineralization and promote remineralization of the teeth;
(e). inhibit microbial biofilm formation in the oral cavity;
(f). reduction or inhibition of gingivitis;
(g). reduction of gingival inflammation;
(h). reduction of inflammation in the oral cavity;
(i). treatment or prevention of dental implant infections;
(j). promotion of healing of sores or cuts in the mouth;
(k). treatment or prevention of xerostomia (dry mouth);
(l). reduction of levels of acid producing bacteria, in the oral cavity;
(m). increase relative levels of non-cariogenic and/or non-plaque forming bacteria;
(n). increasing of relative levels of arginolytic bacteria;
(o). inhibition of microbial biofilm formation in the oral cavity,
(p). raising and/or maintenance of plaque pH at levels of at least pH 5.5 following sugar (e.g. fructose, glucose, lactose and/or sucrose) challenge;
(q). reducing tooth erosion;
(r). immunization of teeth against cariogenic bacteria; and/or
(s). promotion of systemic health, including cardiovascular health, e.g., by reducing potential for systemic infection via the oral tissues.

In a further embodiment of the invention, there is provided a composition of the invention for use in a disease disorder or condition, selected from one or more of the following:
(a). treatment or prevention of erosive tooth demineralization;
(b). reduction or inhibition of formation of dental caries;
(c). reduction or inhibition of gingivitis;
(d). reduction of inflammation in the oral cavity;
(e). treatment or prevention of dental implant infections;
(f). promotion of healing of sores or cuts in the mouth; and/or
(g). treatment or prevention of xerostomia (dry mouth).

In a further embodiment, there is provided a composition of the invention for use in the treatment of mouth, gum and/or palate inflammation.

### Additional/Combination Therapy

Compositions comprising a compound of Table 1 or Table 1a, or salt, derivative or prodrug thereof; may comprise one of more further active agents, for use in the treatment or prevention of diseases, disorders or conditions, or for administration to generally health subjects.

Any active agent which is known to be useful, or which has been used or is currently being used for the treatment or prevention of diseases, disorders or conditions or would benefit a generally healthy subject can be used with a combination of the invention. See, e.g., Gilman et al, Goodman and Gilman's: The Pharmacological Basis of Therapeutics, 14th ed., McGraw-Hill, New York, 2023; The Merck Manual of Diagnosis and Therapy, Robert S. Porter, M. D. et al. (eds.), 20th Ed., Merck Sharp & Dohme Research Laboratories, Rahway, NJ, 2018; Cecil Textbook of Medicine, 27th Ed., Goldman and Schafer (eds.), Elsevier, 2023, and Physicians' Desk Reference (71st ed. 2016) for information regarding therapies (e.g., prophylactic or therapeutic agents) which have been or are currently being used for the treatment or prevention of diseases, disorders or conditions associated with brain health and/or immune health.

In a further embodiment of the invention there is provided a composition, comprising:
(a) a compound of Table 1 or Table 1a, or salt, derivative or prodrug thereof;
(b) One or more agents selected from
   (i) An autophagy enhancer; including an enhancer of specific types of autophagy, including:
      (a). Mitophagy
      (b). Lysophagy
      (c). Aggrephagy
      (d). ERphagy
      (e). Peroxiphagy
      (f). Chaperone-Mediated Autophagy (CMA);
   (ii) an autophagy inducer; and
   (iii) a mitochondrial biogenesis promoting agent; and
   (iv) a senolytic agent.

Examples of autophagy inducers include, but not limited to, carbamazepine, clonidine, lithium, metformin, rapamycin (and rapalogs), rilmenidine, sodium valproate, verapamil, trifluoperazine, statins, tyrosine kinase inhibitors (for example, Akt-mTOR signaling inhibitors and beclin 1 tyrosine phosphorylation inhibitors), BH3 mimetics, caffeine, omega-3 polyunsaturated fatty acids, resveratrol, spermidine, vitamin D pterostilbene, fisetein, genistein, quercetin, kaempferol, minoxidil, actinonin, kinetin triphosphate, pifithrin-a, deferiprone, 1,10′-phenanthroline and trehalose, for example trehalose.

Examples of mitochondrial biogenesis promoting agents include, but are not limited to, PPAR-PGC-1a axis activators (for example, bezafibrate), AMPK activators (for example, resveratrol), Sirt1 agonists (for example, quercetin, resveratrol), anti-oxidants (such as L carnitine, coenzyme Q10, MitoQ10 and other mitochondria-targeted antioxidants, N acetylcysteine (NAC), vitamin C, vitamin E vitamin K1, vitamin B, sodium pyruvate and α-lipoic acid) and NAD precursors (for example, niacinamide).

In a further embodiment, there is provided a composition comprising:
(a) a compound of Table 1 or Table 1a, or salt, derivative or prodrug thereof, for example, norartocarpetin, or salt, derivative or prodrug thereof, and
(b) co-enzyme Q₁₀.

Examples of senolytic agents include luteolin, piperlongumine, epigallocatechin gallate, galangin, apigenin, fisetin and curcumin.

In a further embodiment, there is provided a composition comprising
(a). a compound of Table 1 or Table 1a, or salt, derivative or prodrug thereof; and a compound selected from one or more of the compounds comprising omega-3 polyunsaturated fatty acids, vitamin D, trehalose, N acetylcysteine (NAC), sodium pyruvate and α-lipoic acid and amino acids.

In a further embodiment of the invention there is provided a composition, comprising:
(a) a compound of Table 1 or Table 1a, or salt, derivative or prodrug thereof; and
(b). creatine.

In a further embodiment, there is provided a composition comprising
(a). a compound of Table 1 or Table 1a, or salt, derivative or prodrug thereof; and
(b). fisetin.

In a further embodiment of the invention there is provided a composition, comprising:
(a) a compound of Table 1 or Table 1a, or salt, derivative or prodrug thereof; and
(b) resveratrol.

In a further embodiment of the invention there is provided a composition, comprising:
(a) a compound of Table 1 or Table 1a, or salt, derivative or prodrug thereof; and
(b) One or more agents selected from:
   (i) one or more agents which promote skin regeneration, for example, ascorbic acid or derivatives thereof (such as ascorbic acid glycoside (ascorbyl glucoside)), promatriciel peptides (such as Peptide GHK CU, PeptiYouth™, acetyl hexapeptide 8; and/or copper peptides, and/or tripeptide-1) retinoids, alpha hydroxy acids (such as glycolic or lactic acid, gluconolactone); and
   (ii) one or more skin hydrating agents, for example, hyaluronic acid and fragments thereof, glycerine, Urea, sodium PCA and sodium lactate.

### Cosmetic, Nutraceutical and Pharmaceutical Compositions

For the purposes of administration, in certain embodiments, a compound of Table 1 or Table 1a, or salt, derivative or prodrug thereof described herein are administered as a raw chemical or are formulated as cosmetic, nutraceutical or pharmaceutical compositions. Cosmetic, nutraceutical and pharmaceutical compositions of the present invention comprise an effective amount of a compound of Table 1 or Table 1a, or salts, derivatives or prodrugs thereof. The compound of Table 1 or Table 1a, or salt, derivative or prodrug thereof is present in the composition in an amount which is effective to treat a particular disease or condition of interest, or to enhance health in a generally healthy individual. The activity of a compound of Table 1 or Table 1a, or salt, derivative or prodrug thereof can be determined by one skilled in the art, for example, as described herein. Appropriate effective concentrations and dosages can be readily determined by one skilled in the art.

Administration of a compound of Table 1 or Table 1a, or salt, derivative or prodrug thereof in pure form or in an appropriate cosmetic, nutraceutical or pharmaceutical composition, can be carried out via any of the accepted modes of administration of agents for serving similar utilities. The cosmetic, nutraceutical or pharmaceutical compositions of the invention can be prepared by combining a compound of Table 1 or Table 1a, or salt, derivative or prodrug thereof with an appropriate pharmaceutically acceptable excipient, and may be formulated into preparations in solid, semi-solid, liquid or gaseous forms, such as tablets, capsules, powders, granules, ointments, solutions, suppositories, injections, inhalants, gels, microspheres, and aerosols. The cosmetic, nutraceutical or pharmaceutical compositions of the invention can be prepared by combining a compound of Table 1 or Table 1a, or salt, derivative or prodrug thereof with an appropriate pharmaceutically acceptable excipient, and may be formulated into preparations in solid, semi-solid, liquid or gaseous forms, such as solid dispersions and solid solutions. Typical routes of administering such pharmaceutical compositions include, without limitation, oral, topical, transdermal, inhalation, parenteral, sublingual, buccal, rectal, vaginal, and intranasal. Typical routes of administering such cosmetic compositions include, without limitation, oral, topical, transdermal, sublingual, and buccal. In a specific embodiment, the pharmaceutical composition is a tablet. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a salt of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of a compound of Table 1 or Table 1a or salt, derivative or pro-drug thereof for treatment of a disease or condition of interest in accordance with the teachings of this invention, or administration to a generally healthy person.

The cosmetic, nutraceutical or pharmaceutical compositions of a compound of Table 1 or Table 1a, or salt, derivative or prodrug thereof may be prepared by methodology well known in the pharmaceutical art. For example, a pharmaceutical composition intended to be administered by injection can be prepared by combining a compound of Table 1 or Table 1a, or salt, derivative or prodrug thereof of the invention with sterile, distilled water so as to form a solution. A surfactant or other solubilizing excipient may be added to facilitate the formation of a homogeneous solution or suspension.

Surfactants are compounds that non-covalently interact with the salt of the invention so as to facilitate dissolution or homogeneous suspension of the compound in the aqueous delivery system.

In other embodiments, a solid cosmetic, nutraceutical or pharmaceutical composition intended for oral administration can be prepared by mixing an effective amount of a compound of Table 1 or Table 1a, or salt, derivative or prodrug thereof with at least one suitable nutraceutically or pharmaceutically acceptable excipient to form a solid pre-formulation composition, which then may be readily subdivided into equally effective unit dosage forms such as tablets, pills and capsules. Accordingly, in some embodiments, a cosmetic, nutraceutical or pharmaceutical composition is provided, which includes a effective amount of a compound of Table 1 or Table 1a, or salt, derivative or prodrug thereof.

The Compounds of Table 1 or Table 1a, or salts, derivatives or prodrugs thereof of the invention are administered in an effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. In some embodiments, the compounds of Table 1 or Table 1a, or salts, derivatives or prodrugs thereof, can be administered alone or in combination with other agents one time a day, or two times a day, or three times a day, or four times a day, for as long as required by the patient or subject (e.g. for multiple years, months, weeks, or days).

In one embodiment, the composition is administered in the form of a composition comprising: a) a medium-chain triglyceride; b) one or more compounds of Table 1 or Table 1a, or salts, derivatives or prodrugs thereof.

The administration of compositions of the present invention may involve topical administration of a composition comprising a compound of Table 1 or Table 1a, or a salt, derivative or prodrug thereof, to a subject. In one embodiment, compositions comprise concentrations of compounds of Table 1 or Table 1a, or a salt, derivative or prodrug thereof, are in the range of about 0.1% (w/w) to about 5% (w/w) of the composition, such as about 0.25% (w/w) to about 5% (w/w), for example, about 0.5% (w/w) to about 4% (w/w), for example. About 0.5% (w/w) to about 3% (w/w), or about 0.5% (w/w) to about 2% (w/w) or about 1% (w/w) to about 2% (w/w), for example, about 1% (w/w), about 1.5% (w/w) or about 2% (w/w).

The administration of compositions of the present invention may involve oral administration of a composition comprising a compound of Table 1 or Table 1a, or a salt, derivative or prodrug thereof, to a subject. In one embodiment, compositions comprise concentrations of compounds of Table 1 or Table 1a, or a salt, derivative or prodrug thereof, are in the range of about 0.1% (w/w) to about 5% (w/w) of the composition, such as about 0.25% (w/w) to about 5% (w/w), for example, about 0.5% (w/w) to about 4% (w/w), for example. About 0.5% (w/w) to about 3% (w/w), or about 0.5% (w/w) to about 2% (w/w) or about 1% (w/w) to about 2% (w/w), for example, about 1% (w/w), about 1.5% (w/w) or about 2% (w/w).

In one embodiment, concentrations of compounds of Table 1 or Table 1a, or a salt, derivative or prodrug thereof, are in the range of about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1.0% (w/w), about 1.2% (w/w), about 1.4% (w/w), about 1.6% (w/w), about 1.8% (w/w), about 2.0% (w/w), about 2.2% (w/w), about 2.4% (w/w), or about 2.6% (w/w),

The administration of compositions of the present invention may involve oral administration of a compound of Table 1 or Table 1a, or a salt, derivative or prodrug thereof, to a subject. Typically, an oral dose of a composition of the invention comprises from about 50 mg to about 2500 mg, for example, about 125 mg to about 2500 mg, about 125 mg to about 2000 mg, about 250 mg to about 2500 mg, about 500 mg to about 2000 mg, about 250 mg to about 1500 mg, such as about 250 mg to about 1000 mg of the compound of Table 1 or Table 1a, or a salt, derivative or prodrug thereof. In a further embodiment an oral dose of a composition of the invention may comprise from about 125 mg to 2500 mg of Table 3 or 3a, or a salt, derivative or prodrug thereof. For example, about 50 mg, about 75 mg, about 100 mg, about 125 mg, about 250 mg, 500 mg, about 1000 mg, about 1250 mg, about 1500 mg, about 1750 mg about 2000 mg, about 2250 mg or about 2500 mg of a compound of Table 1 or Table 1a, or a salt, derivative or prodrug thereof.

The administration of compounds of the present invention may involve oral administration of a compound of Table 1 or Table 1a, or a salt, derivative or prodrug thereof, to a subject. For oral use, a compound of Table 1 or Table 1a, or a salt, derivative or prodrug thereof will generally be administered in an amount ranging from about to 0.2-150 milligram (mg) per kilogram (kg) of body weight of the subject. In one embodiment, the compound of Table 1 or Table 1a, or a salt, derivative or prodrug thereof, is administered in a dose equal or equivalent to 2-120 mg per kg body weight of the subject. In one embodiment, a compound of Table 1 or Table 1a, or a salt, derivative or prodrug thereof, is administered in a dose equal or equivalent to 4-90 mg per kg body weight of the subject. In one embodiment, a compound of Table 1 or Table 1a, or a salt, derivative or prodrug thereof, is administered in a dose equal or equivalent to 8-30 mg per kg body weight of the subject. Where a precursor of a compound of Table 1 or Table 1a, or a salt, derivative or prodrug thereof is to be administered rather than a compound of Table 1 or Table 1a, it is administered in an amount that is equivalent to the above-stated amounts of the compound of Table 1 or Table 1a, or a salt, derivative or prodrug thereof.

In a further embodiment of the invention, compositions of the invention include oral care compositions. Examples of oral care compositions include toothpaste, powder, mouthwash, oral sprays, oral gels, mouth rinses, chewing gum, and lozenges, for example, toothpaste, oral gels or mouthwash, for example toothpaste.

According to a further embodiment of the invention, there is provided an oral care composition of the invention, wherein the composition is applied to an oral care product.

According to a further embodiment of the invention, there is provided an oral care composition of the invention, wherein the composition is incorporated into the matrix of an oral care product.

Examples of oral care products include dental floss, dental tape, dental strips and dental sticks (tooth picks).

According to one aspect of the invention there is provided an oral care composition comprising:
a) a compound of Table 1 or Table 1a, or a salt, prodrug or derivative thereof; and
b) an oral care composition additive, wherein the oral care composition additive is selected from one or more of a fluoride source, a fluoride replacement, an antiseptic and a whitening agent.

According to a further aspect of the invention, there is provided an oral care composition, comprising:
a) a compound of Table 1 or Table 1a, or a salt, prodrug or derivative thereof; and
b) a fluoride source.

Examples of suitable fluoride sources, include fluoride salts, for example, sodium fluoride, stannous fluoride, potassium fluoride, potassium stannous fluoride, indium fluoride, zinc fluoride, ammonium fluoride, stannous chlorofluoride, sodium monofluorophosphate, sodium fluorosilicate, ammonium fluorosilicate, amine fluoride, titanium fluoride, hexafluorosulphate, or a mixture of two or more thereof. Further examples of suitable fluoride sources can be found in U.S. Pat. Nos. 3,535,421, 4,885,155, and 3,678,154.

Examples of further fluoride sources include sodium fluoride, potassium fluoride, disodium monofluorophosphate, tin (II) fluoride, dipotassium fluorophosphates, calcium fluorophosphates, calcium fluoride, ammonium fluoride, aluminium fluoride, hexadecyl ammonium fluoride, 3-(N-hexadecyl-N-2-hydroxy-ethylammonio) ammonium difluoride, N,N-N-Tris(polyoxyethylene)-N-hexadecyl-propylenediaminedihydrofluoride disodium hexafluorosilicate, dipotassiumhexafluorosilicate, ammonium hexafluorosilicate, magnesium hexafluorosilicate or ammonium fluorophosphates, or any combinations of two or more thereof.

More commonly used fluoride sources s are typically sodium fluoride, sodium monofluorophosphate, and stannous fluoride. Sodium fluoride and sodium monofluorophosphate are effective sources of fluoride ions that remineralize and strengthen weakened enamel thus allowing fighting cavities. By comparison, stannous fluoride not only delivers cavity fighting fluoride, but it also has antibacterial properties, and it provides an anti-sensitivity mechanism of action. Stannous fluoride has both bactericidal and bacteriostatic properties, which fight plaque and treat/prevent gingivitis. Stannous fluoride also deposits a protective mineral barrier over exposed dentinal tubules to help prevent sensitivity pain from triggers such as hot or cold liquids and foods.

According to a further aspect of the invention, there is provided an oral care composition, essentially free of a fluoride source.

According to a further aspect of the invention, there is provided a oral care composition, comprising:
a) a compound of Table 1 or Table 1a, or a salt, prodrug or derivative thereof;
b) a fluoride source; and
c) an abrasive.

According to a further aspect of the invention, there is provided an oral care composition, comprising:
a) a compound of formula (l) or a salt thereof, as defined herein; and
b) a fluorine replacement.

Examples of fluoride replacements include arginine, xylitol, citric acid, zinc citrate, activated charcoal, calcium sodium phosphosilicate (Novamin), casein phosphopeptide-amorphous calcium phosphate (CPP-ACP), and hydroxyapatite, for example, calcium hydroxyapatite.

According to a further aspect of the invention, there is provided an oral care composition, comprising:
a) a compound of Table 1 or Table 1a, or a salt, prodrug or derivative thereof, as defined herein; and
b) an antiseptic.

Examples of antiseptics include thymol, eucalyptol, menthol, and/or methyl salicylate.

In a further embodiment, an antiseptic is selected from one or more of essential oils (e.g., rosemary extract, tea extract, magnolia extract, thymol, menthol, eucalyptol, geraniol, carvacrol, citral, hinokitol, miswak extract, sea-buckthorn extract), and/or one or more zinc salts.

In a further embodiment the antiseptic is menthol.

According to a further aspect of the invention, there is provided an oral care composition, comprising:
a) a compound of Table 1 or Table 1a, or a salt, prodrug or derivative thereof; and
b) a whitening agent.

Examples of whitening agents include a peroxide source, such as hydrogen peroxide, sodium peroxide and/or carbamide peroxide.

Any given dose may be given as a single dose or as divided doses.

The compositions of the invention can be taken as a single treatment or, more commonly, as a series of treatments. In one example, a subject takes a dose before or after exercise. For a subject who is not able to exercise, a dose of the composition may, for example, be taken once, twice or three times per day, or one, two, three, four, five or six times per week. In another example, the intervention may be taken by a subject independent of the subject's ability or need to exercise. It will also be appreciated that the effective dosage of the compound may increase or decrease over the course of a particular treatment.

Compositions of the invention comprise both oral and topical formulations. In one embodiment ranges for the administration of compounds of Table 1 or Table 1a in compositions of the invention are found in Table 3 below. In this table the limits of the range may be varied by including:
(c) a variation of ±20% of the relevant value, for example ±15% of the relevant value, such as ±10% of the relevant value or ±5% of the relevant value; and/or
(d) narrower ranges within the range below, centring on the midpoint, for example:
   ±about 45% of the midpoint
   ±about 40% of the midpoint
   ±about 35% of the midpoint
   ±about 30% of the midpoint
   ±about 25% of the midpoint
   ±about 20% of the midpoint
   ±about 15% of the midpoint
   ±about 10% of the midpoint
   ±about 55% of the midpoint

For example, for a range of 0-100, ±about 45% equates to the range 5 to 95.

In a further embodiment, there is provided a composition comprising a compound of Table 1 or Table 1a, wherein the composition is a topical composition, for example, a topical composition for skin and/or hair wherein the composition comprises about 0.1% (w/w) to about 5% (w/w) of a compound of Table 1 or Table 1a, or a salt, derivative or prodrug thereof, for example, about 0.1% (w/w) to about 4% (w/w), about 0.1% (w/w) to about 3% (w/w), about 0.1% (w/w) to about 2.5% (w/w), about 0.1% (w/w) to about 2% (w/w), about 0.1% (w/w) to about 1.5% (w/w), about 0.1% (w/w) to about 1.25% (w/w), or about 0.1% (w/w) to about 1% (w/w) of a compound of Table 1 or Table 1a, or a salt, derivative or prodrug thereof, such as about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 0.9% (w/w), about 1.0% (w/w), about 1.25% (w/w), about 1.5% (w/w), about 1.75% (w/w) or about 2.0% (w/w) of a compound of Table 1 or Table 1a, or a salt, derivative or prodrug thereof.

TABLE 3

| | Oral dose | Oral dose | Topical | Topical |
|---|---|---|---|---|
| | Lower | Higher | Lower | Higher |
| | limit of | limit of | limit of | limit of |
| | range | range | range | range |
| | (mg) | (mg) | (%)(w/w)¹ | (%)(w/w)¹ |
| Norartocarpetin | 25 | 2500 | 0.019 | 2.0 |
| Chrysoeriol | 25 | 2500 | 0.035 | 2.0 |
| Juglanin | 25 | 2500 | 0.051 | 2.561 |
| Meisoindigo | 25 | 2500 | 0.069 | 3.431 |
| Byakangelicol | 25 | 2500 | 0.075 | 3.762 |
| N-Nornuciferine | 25 | 2500 | 0.092 | 4.586 |
| 3′-Demethylnobiletin | 25 | 2500 | 0.093 | 4.666 |
| 3-O-Methylquercetin | 25 | 2500 | 0.108 | 5.389 |

TABLE 3a

| | Oral | Oral | Topical | Topical |
|---|---|---|---|---|
| | dose | dose | Lower | Higher |
| | Lower | Higher | limit of | limit of |
| | limit of | limit of | range | range |
| | range | range | (%) | (%) |
| | (mg) | (mg) | (w/w)¹ | (w/w)¹ |
| Norartocarpetin | 19 | 385 | 0.019 | 0.963 |
| Chrysoeriol | 35 | 707 | 0.035 | 1.768 |
| Juglanin | 51 | 1025 | 0.051 | 2.561 |
| Meisoindigo | 69 | 1373 | 0.069 | 3.431 |
| Byakangelicol | 75 | 1505 | 0.075 | 3.762 |
| N-Nornuciferine | 92 | 1835 | 0.092 | 4.586 |
| 3′-Demethylnobiletin | 93 | 1867 | 0.093 | 4.666 |
| 3-O-Methylquercetin | 108 | 2155 | 0.108 | 5.389 |
| Dehydrocorydaline | 129 | 2588 | 0.129 | 6.471 |
| (hydroxyl) | | | | |
| Pachypodol | 131 | 2612 | 0.131 | 6.531 |
| 4′,5-Dihydroxyflavone | 137 | 2742 | 0.137 | 6.854 |
| 5-Hydroxyflavone | 140 | 2799 | 0.140 | 6.998 |
| Scutellarein tetramethyl | 143 | 2868 | 0.143 | 7.169 |
| ether | | | | |
| Isosinensetin | 144 | 2878 | 0.144 | 7.194 |
| Robustine | 156 | 3112 | 0.156 | 7.780 |
| Skullcapflavone II | 165 | 3304 | 0.165 | 8.260 |
| Dehydrocorydaline | 167 | 3331 | 0.167 | 8.327 |
| (nitrate) | | | | |
| Coptisine (chloride) | 180 | 3598 | 0.180 | 8.994 |
| Mosloflavone | 182 | 3639 | 0.182 | 9.099 |
| Iristectorin B | 184 | 3682 | 0.184 | 9.204 |
| Chrysosplenol D | 186 | 3711 | 0.186 | 9.278 |
| Phellopterin | 188 | 3763 | 0.188 | 9.407 |
| Chrysosplenetin | 190 | 3793 | 0.190 | 9.484 |
| Angelicin | 196 | 3918 | 0.196 | 9.794 |
| Dehydrocorydaline | 196 | 3927 | 0.196 | 9.817 |
| (chloride) | | | | |

¹A lotion or ointment etc. with the indicated dose of active ingredient.

In a further embodiment, there is provided a composition comprising a compound of Table 3 or 3a, or a salt, derivative or prodrug thereof, wherein the composition comprises a compound of Table 3 or 3a or a salt, derivative or prodrug thereof, in the range recited in Table 3 or 3a. In a further embodiment, the composition comprises about 25 mg to about 2500 mg, for example, about 125 mg to about 2500 mg, about 125 mg to about 2000 mg, about 250 mg to about 2500 mg. about 500 mg to about 2000 mg, about 250 mg to about 1500 mg, such as about 250 mg to about 1000 mg of a compound of Table 3 or 3a, or a salt, derivative or prodrug thereof. In a further embodiment the composition of the invention comprises from about 125 mg to 2500 mg of Table 3 or 3a, or a salt, derivative or prodrug thereof. For example, about 50 mg, about 75 mg, about 100 mg, about 125 mg, about 250 mg, 500 mg, about 1000 mg, about 1250 mg, about 1500 mg, about 1750 mg about 2000 mg, about 2250 mg or about 2500 mg of a compound of Table 3 or 3a, or a salt, derivative or prodrug thereof.

### Articles of Manufacture and Kits

Compositions comprising a compound of Table 1 or Table 1a, or salt, derivative or prodrug thereof can be prepared, placed in an appropriate container, and labelled for treatment of an indicated condition. Accordingly, there also is contemplated an article of manufacture, such as a container comprising a dosage form of one or more of the crystalline forms described herein and a label containing instructions for use of the compound

In some embodiments, the article of manufacture is a container comprising a dosage form of one or more of the Compounds of Table 1 or Table 1a, or salts, derivatives or prodrugs thereof, and one or more pharmaceutically acceptable excipients or other ingredients. In some embodiments of the articles of manufacture described herein, the dosage form is a solution.

Kits also are contemplated. For example, a kit can comprise a dosage form of a pharmaceutical composition and a package insert containing instructions for use of the composition in treatment of a medical condition for administration to a healthy subject. In a further embodiment a kit may comprise multiple individual dosage forms, each comprising a effective amount of a compound of Table 1 or Table 1a, or salt, derivative or prodrug thereof, and instructions for their administration to a human in need thereof. Each of the individual dosage forms may comprise any effective amount of a compound of Table 1 or Table 1a, or salt, derivative or prodrug thereof in combination with at least one pharmaceutically effective excipient. The individual dosage forms may be in the form of, as examples, a solution, a tablet, a pill, a capsule, a sachet, a sublingual medicament, a lyophilized powder, a spray-dried powder, or a liquid composition for oral, parenteral, or topical administration. The instructions for use in the kit may be for treating a disease described here in or for administration to a healthy subject. The instructions may be for prophylaxis or the treatment of a disease described herein or for promoting healthy aging or improving muscle function.

### Animals

Compositions of the invention are suitable for other mammals in addition to humans, scaled appropriately to the size of the non-human mammal. For example, scaled according to the following table;

| | Reference | To convert dose | To convert human dose in mg/kg | |
|---|---|---|---|---|
| | body | in mg/kg to | to AED in mg/kg. either | |
| | weight | dose in mg/m2. | Multiply human | Divide human |
| Species | (kg) | divide by km | dose by | dose by |
| Mouse | 0.02 | 3 | 12.3 | 0.081 |
| Hamster | 0.08 | 5 | 7.4 | 0.135 |
| Rat | 0.15 | 6 | 6.2 | 0.162 |
| Ferret | 0.3 | 7 | 5.3 | 0.189 |
| Guinea Pig | 0.4 | 8 | 4.6 | 0.216 |
| Rabbit | 1.8 | 12 | 3.1 | 0.324 |
| Cat | 2 | 31.7 | 3.2 | 0.316 |
| Monkey | 3 | 12 | 3.1 | 0.324 |
| Dog | 10 | 20 | 1.9 | 0.541 |
| Marmoset | 9.35 | 6 | 6.2 | 0.162 |
| Squireel | 0.6 | 7 | 5.3 | 0.189 |
| Monkey | | | | |
| Baboon | 12 | 20 | 1.9 | 0.541 |
| Micro pig | 12 | 27 | 1.4 | 0.73 |
| Mini pig | 40 | 35 | 1.1 | 0.946 |

Therefore, according to a further aspect of the invention there is provided a composition comprising a compound of Table 1 or Table 1a, or a salt, derivative or prodrug thereof, for use in a use of the invention as defined above in a non-human mammal wherein the dose is scaled according to the table above.

### Definitions

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment.

Furthermore, the particular features, structures, or characteristics may be combined in any suitable manner in one or more embodiments.

Embodiments that reference throughout this specification to “a compound” includes the crystalline, salt, co-crystal, and solvate forms of the formulas and/or compounds disclosed herein.

The term ‘about’ refers to a tolerance of ±20% of the relevant value, for example ±15% of the relevant value, such as ±10% of the relevant value or ±5% of the relevant value.

The term “elite” athlete may refer to a human subject who has a VO2max of greater than 65 mL Kg−1 min−1, a 3 km running personal best time below 9 minutes, or both.

References to norartocarpetin (2′,4′,5,7-tetrahydroxyflavone) refers to the norartocarpetin compound purified from natural sources or obtained by chemical synthesis and refers to extracts comprising norartocarpetin, for example, extracts of material from plants of the Moraceae family, particularly species belonging to the genus Artocarpus. Furthermore, references to norartocarpetin refers to extracts from biological sources, such as Artocarpus heterophyllus, Artocarpus communis, Artocarpus lakoocha, Artocarpus dadah, and other species within the genus Artocarpus and related taxa.

In a further embodiment, norartocarpetin comprises norartocarpetin derived from Artocarpus heterophyllus and Artocarpus communis, which exhibit high concentrations of the compound in their wood, bark, and stem tissues, or extracts derived therefrom.

In a further embodiment, norartocarpetin comprises norartocarpetin derived from Artocarpus communis, Artocarpus dadah, Artocarpus fretessii, Artocarpus heterophyllus, Artocarpus integer, Artocarpus kemando, Artocarpus lakoocha and Artocarpus scortechinii.

In a further embodiment, norartocarpetin comprises norartocarpetin derived from species of the genus Morus, including Morus alba, Morus nigra, and Morus rubra, or extracts derived therefrom. These species, commonly known as mulberry, are cultivated widely and their leaves, roots, and hairy root cultures have been demonstrated to contain norartocarpetin.

The term “Pharmaceutically acceptable excipient” includes without limitation any adjuvant, carrier, excipient, glidant, sweetening agent, diluent, preservative, dye/colorant, flavour enhancer, surfactant, wetting agent, dispersing agent, suspending agent, stabilizer, isotonic agent, solvent, and/or emulsifier, or a combination of one or more of the above which has been approved by the United States Food and Drug Administration, or equivalent national drug regulatory body, as being acceptable for use in humans or domestic animals.

The term “pharmaceutical composition” refers to a formulation of a salt of the invention and a medium generally accepted in the art for the delivery of the biologically active salt to mammals, e.g., humans. Such a medium includes all pharmaceutically acceptable excipients therefor.

The term ‘co-enzyme Q10 refers to the compound in any one of its redox states, for example, fully oxidized (ubiquinone), semiquinone (ubisemiquinone), and fully reduced (ubiquinol).

The terms “Effective amount” or “therapeutically effective amount” refers to an amount of a compound according to the invention, which when administered to a subject or patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the invention which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the invention, and the age, body weight, general health, sex and diet of the patient. Such a therapeutically effective amount can be determined routinely by one of ordinary skill in the art having regard to their own knowledge, the state of the art, and this disclosure.

The terms “subject” or “patient” refer to an animal, such as a mammal (including a human), that has been or will be the object of treatment, observation or experiment. The methods described herein may be useful in human therapy and/or veterinary applications. In some embodiments, the subject is a mammal (or the patient). In some embodiments the subject (or the patient) is human, domestic animals (e.g., dogs and cats), farm animals (e.g., cattle, horses, sheep, goats and pigs), and/or laboratory animals (e.g., mice, rats, hamsters, guinea pigs, pigs, rabbits, dogs, and monkeys). In some embodiments, the subject (or the patient) is a human. “Human (or patient) in need thereof refers to a human who may have or is suspect to have diseases or conditions that would benefit from certain treatment; for example, being treated with the compounds disclosed herein according to the present application.

The term “Unit dosage forms” are physically discrete units suitable as unitary dosages for subjects (e.g., human subjects and other mammals), each unit containing a predetermined quantity of active material calculated to produce the desired therapeutic effect, in association with a suitable pharmaceutical excipient.

Each of the references including all patents, patent applications and publications cited in the present application is incorporated herein by reference in its entirety, as if each of them is individually incorporated. Further, it would be appreciated that, in the above teaching of invention, the skilled in the art could make certain changes or modifications to the invention, and these equivalents would still be within the scope of the invention defined by the appended claims of the application. Each of the references including all patents, patent applications and publications cited in the present application is incorporated herein by reference in its entirety, as if each of them is individually incorporated. Further, it would be appreciated that, in the above teaching of invention, the skilled in the art could make certain changes or modifications to the invention, and these equivalents would still be within the scope of the invention defined by the appended claims of the application.

### Example 1: Effect of Test Compounds on Mitochondrial Function

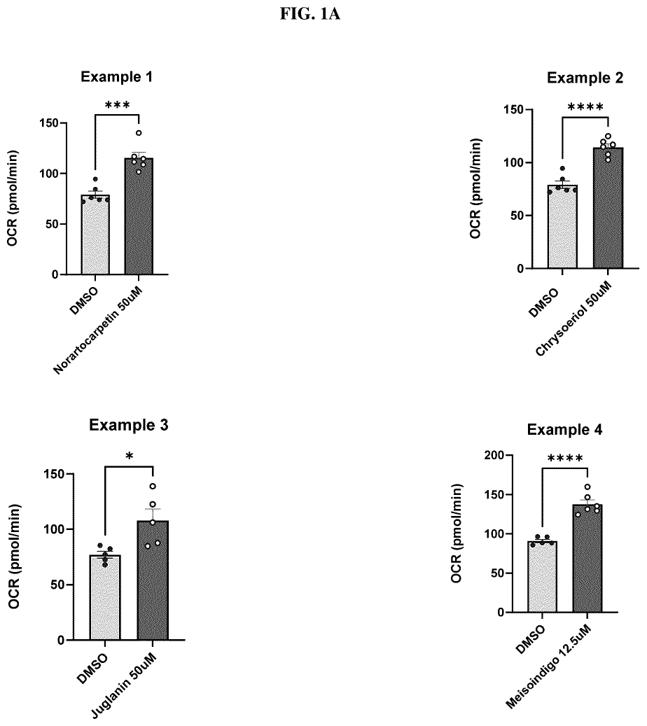

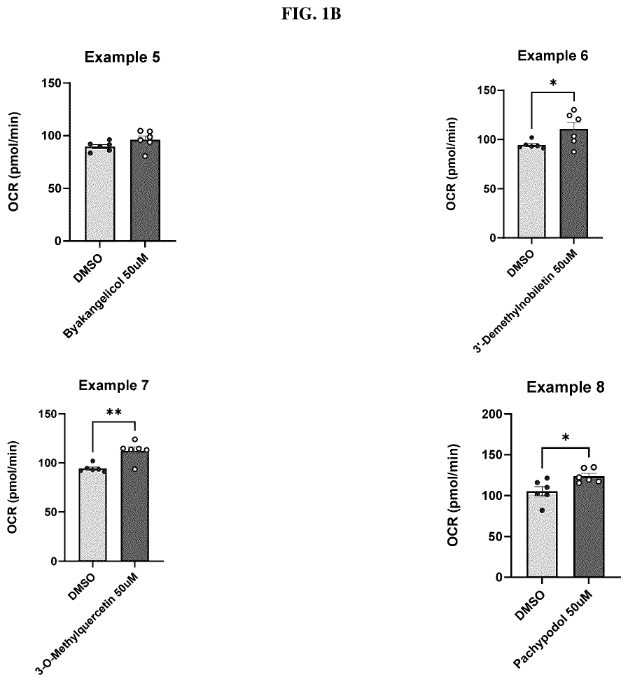

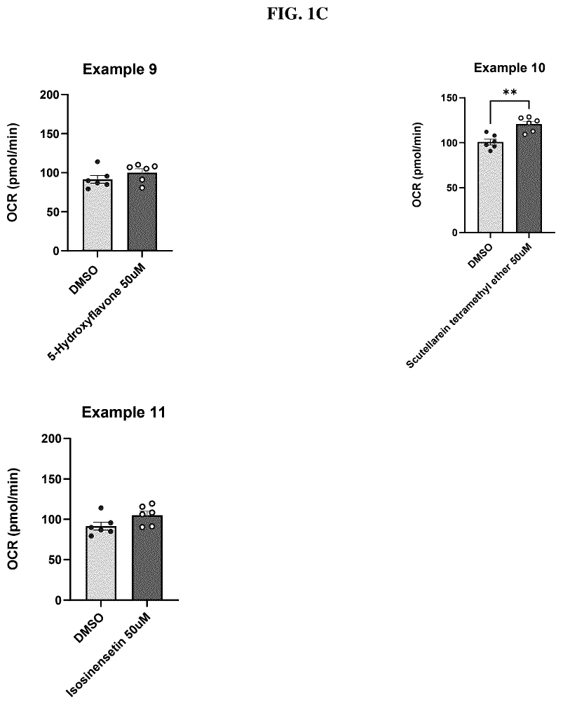

We tested different concentrations of test compounds by mitochondrial respirometry (Seahorse, Agilent). Mouse muscle C2C12 myotubes have been treated with either a test compound (sourced from MedChemExpress, Monmouth Junction, New Jersey, USA) at the indicated dose or DMSO as control for 24 hours. FIGS. 1a to 1c shows the impact of compounds on maximal respirations in C2C12, measured as oxygen consumption rate per minute (OCR, pmol/min). All compounds induced maximal mitochondrial respiration to various degrees. Table 4 shows the percentage increase in mitochondrial respiration of test compounds. Enhanced maximal mitochondrial respiration indicates improved mitochondrial capacity and efficiency, which is associated with healthier, more metabolically active cells. In skeletal muscle cells, such as C2C12 myotubes, this translates to better energy metabolism and resilience to stress. Skeletal muscle cells are a well-established model for studying mitochondrial activity due to their high energy demands.

Mitochondrial responses observed in C2C12 myotubes are indicative of similar effects in other mitochondria-rich cell types where mitochondrial function is essential, including immune, neuronal, cardiac, and epithelial cells. Consequently, findings from this model are broadly translatable and relevant to systemic mitochondrial health. These insights have significant implications for improving conditions associated with impaired mitochondrial function across multiple tissues.

TABLE 4

| | | | % increase in |
|---|---|---|---|
| | | | mitochondrial |
| Example | Structure | Compound Name | respiration |
| 1 | 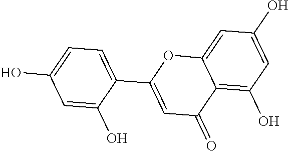 [SMILES: O=c1cc(-c2ccc(O)cc2O)oc2cc(O)cc(O)c12] | Norartocarpetin | +60.4 |
| 2 | 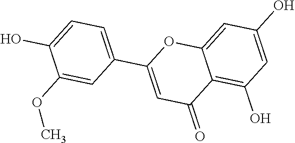 [SMILES: COc1cc(-c2cc(=O)c3c(O)cc(O)cc3o2)ccc1O] | Chrysoeriol | +43.6 |
| 3 | 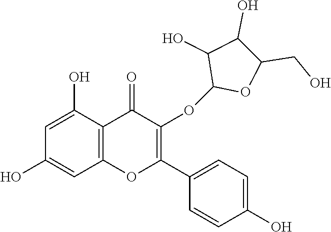 [SMILES: O=c1c(OC2OC(CO)C(O)C2O)c(-c2ccc(O)cc2)oc2cc(O)cc(O)c12] | Juglanin | +40.1 |
| 4 | 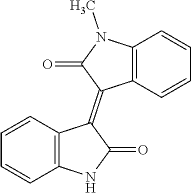 [SMILES: CN1C(=O)/C(=C2/C(=O)Nc3ccccc32)c2ccccc21] | Meisoindigo | +51.4 |
| 5 | 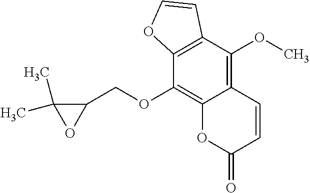 | Byakangelicol | +7.2 |
| 6 | 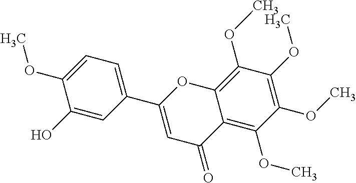 | 3′-Demethylnobiletin | +17.3 |
| 7 | 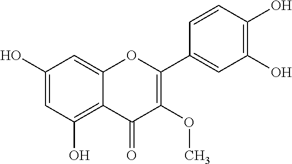 [SMILES: COc1c(-c2ccc(O)c(O)c2)oc2cc(O)cc(O)c2c1=O] | 3-O-Methylquercetin | +19 |
| 8 | 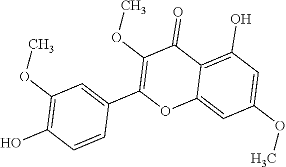 [SMILES: COc1cc(O)c2c(=O)c(OC)c(-c3ccc(O)c(OC)c3)oc2c1] | Pachypodol | +17.4 |
| 9 | 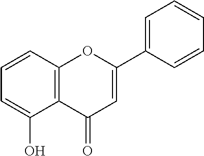 [SMILES: O=c1cc(-c2ccccc2)oc2cccc(O)c12] | 5-Hydroxyflavone | +9.4 |
| 10 | 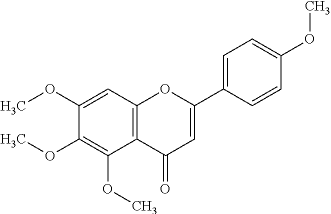 | Scutellarein tetramethyl ether | +19.7 |
| 11 | 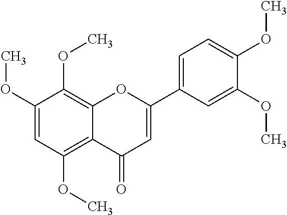 [SMILES: COc1ccc(-c2cc(=O)c3c(OC)cc(OC)c(OC)c3o2)cc1OC] | Isosinensetin | +14.7 |

### Methods

Oxygen consumption was measured using the XF96 Extracellular Flux Analyzer (Agilent). Once fully differentiated in a 96-well plate, C2C12 myotubes were treated in DMEM-based substrate limited medium, containing 0.5 mM glucose, 1 mM GlutaMAX, 0.5 mM carnitine, and either DMSO (as control) or 50 μM of the indicated test compound, for 24 h. 45 min before the assay, cells were washed two times with Assay Medium (111 mM NaCl, 4.7 mM KCl, 1.25 mM CaCl2, 2 mM MgSO4, 1.2 mM NaH2PO4, 2.5 mM glucose, 0.5 mM carnitine, oleic acid 5% and 5 mM HEPES adjusted to pH 7.4 at 37° C. on the day of the assay). Cells were then incubated for 30 min in a non-CO2 incubator. Oxygen consumption rate was determined at maximal level, after the addition of FCCP at 3 μM.

### Example 2

Different concentrations of test compounds from Table 1 or Table 1a are tested by mitochondrial respirometry (Seahorse, Agilent) in primary human skin cells. Human primary dermal fibroblasts (HDF, Promocell,ref. C-12302) are treated with either a test compound (sourced from MedChemExpress, Monmouth Junction, New Jersey, USA) at the indicated dose or DMSO as control for 24 hours. The compounds are assessed for their ability to induce mitochondrial respiration to various degrees following the methods as in Example 1.

### Example 3

We tested different concentrations of test compounds from Table 1 or Table 1a to assess their impact in enhancing the expression of genes associated with improved mitochondrial functions in primary human skin cells. Human primary dermal fibroblasts (HDF) and NHEK (Normal Human Epidermal Keratinocytes) are treated with either a test compound (sourced from MedChemExpress, Monmouth Junction, New Jersey, USA) at the indicated dose or DMSO as control for 24 hours.

RNA from cells is extracted using Rneasy mini kit (Qiagen, 74106) and then transcribed to cDNA by the QuantiTect Reverse Transcription Kit (Qiagen, 205,313) following the manufacturer's instructions. The RT-qPCR reactions is performed using TaqMan master mix (Thermo Fisher 4,369,510). List of TaqMan probes used includes, but is not restricted, to: GAPDH: Hs99999905_m1; RPLP2 Hs01115128_Gh; LC3: Hs00797944_s1; P62/SQSTM1: Hs00177654_m1; PINK1: Hs00260868_m1, COL1A1: HS00164004_m1; IL1b: Hs00174097_m1; IL6: Hs00174131_m1; MMP1: Hs00899658_m1; MMP3: Hs00968305_m1, Park2: Hs01038318_m1. The analysis was performed with Quantstudio 6 Flex (Life Technologies). All quantitative polymerase chain reaction (qPCR) results were presented relative to the mean of housekeeping genes (ΔΔCt method). mRNA levels were normalized over the average GAPDH and RPLP2.

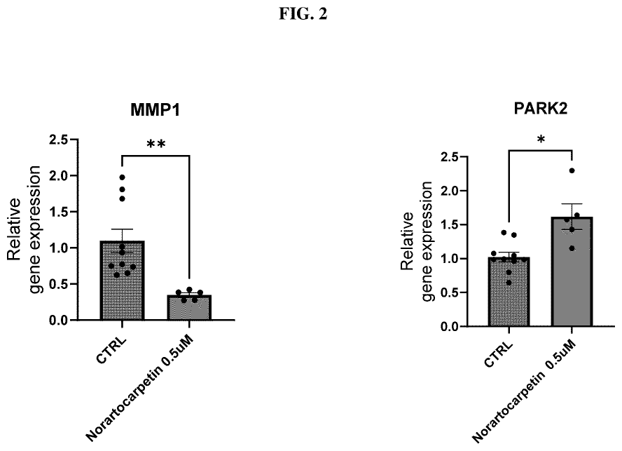

Treatment of human dermal fibroblasts with Norartocarpetin at a concentration of 0.5 μM for 24 hours resulted in significant modulation of biomarkers associated with skin health. As shown in FIG. 2 and Table 5, Norartocarpetin exposure led to a reduction in markers of collagen degradation (metalloproteinase 1, MMP1) and mitophagy enhancement (PINK1).

Reduction of MMP1 (metalloproteinase 1) expression limits collagen degradation in the dermal matrix, thereby supporting improved skin firmness, elasticity, and structural integrity. Upregulation of PINK1 promotes mitophagy and mitochondrial quality control, thereby enhancing cellular energy homeostasis and resilience.

Cells treated with Norartocarpetin exhibited a 68% decrease in MMP1 levels compared to untreated controls, indicating a strong inhibitory effect on collagen breakdown. Norartocarpetin treatment also induced a 58% increase in PARK2 (parkin, mitophagy marker) expression relative to control cells, consistent with enhanced mitophagy activity.

Together, these findings demonstrate that Norartocarpetin exerts a beneficial effect on human dermal fibroblasts by simultaneously reducing collagen degradation and promoting mitophagy. These biomarker changes support the use of Norartocarpetin to improve skin health outcomes.

TABLE 5
Effect of Norartocarpetin on relative gene expression

| | % of | Adjusted |
|---|---|---|
| Comparison | changes | P Value |
| MMP1: Norartocarpetin 0.5 uM vs. DMSO | −68.16% | ** |
| PARK2: Norartocarpetin 0.5 uM vs. DMSO | +58.25% | * |

### Example 4

Different concentrations of test compounds from Table 1 or Table 1a are tested by mitochondrial respirometry (Seahorse, Agilent) in primary human hair cells. Human Hair Follicle Dermal Papilla Cells (HFDPC) are treated with either a test compound (sourced from MedChemExpress) at the indicated dose or DMSO as control for 24 hours. The compounds are assessed for their ability to induce mitochondrial respiration and gene expression to various degrees following the methods as in Example 1 and Example 3.

### Example 5

We tested different concentrations of test compounds from Table 1 or Table 1a in NHEK (Normal Human Epidermal Keratinocytes) to assess their ability to prevent UVA- and UVB-induced cellular inflammation. Cells are pre-treated with either a test compound (sourced from MedChemExpress, Monmouth Junction, New Jersey, USA) at the indicated doses or DMSO as control for 24 hours.

Keratinocytes were seeded in 24-well plates and grown for 24 hours in DMEM with 10% FCS. The medium was then replaced with either control medium or medium containing Norartocarpetin at 1 μM and 5 μM, and cells were incubated for another 24 hours. After this, the medium was changed to assay medium (DMEM with 1% FCS), and cells were irradiated with UVB (275 mJ/cm²) plus UVA (2.1 J/cm²) using a SOL500 Sun Simulator with an H2 filter (Dr. Honle, AG). A non-irradiated control was included in parallel. Following irradiation, the medium was replaced again with either control medium or medium containing the test compounds, and cells were incubated for 48 hours.

Norartocarpetin was assessed for its ability to reduce the release of the pro-inflammatory biomarker prostaglandin E2 (PGE2) in the culture subnatants, measured using a ELISA kit (Enzo, ref. ADI-901-001) according to the supplier's instructions.

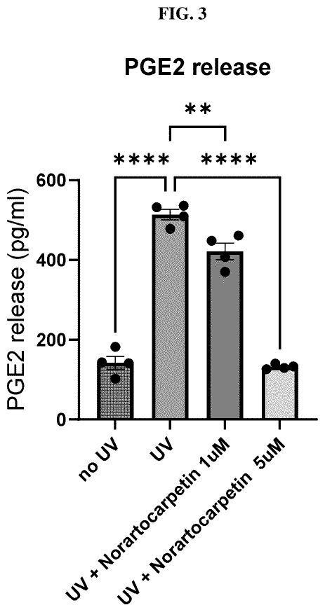

In this experiment, normal human epidermal keratinocytes (NHEK) were treated with Norartocarpetin at concentrations of 1 μM and 5 μM following the protocol indicated above. As shown in FIG. 3 and Table 6, UV exposure led to a strong induction of PGE2 release (+261.79% compared to non-irradiated control. However, treatment with Norartocarpetin significantly reduced this UV-induced increase. At 1 μM, Norartocarpetin lowered PGE2 levels by 17.96%, while at 5 μM the reduction was more pronounced, reaching 74.31%. These results indicate that Norartocarpetin effectively counteracts UV-stimulated PGE2 production in keratinocytes, thereby providing a functional benefits against inflammatory responses, which translates into practical advantages such as preservation of skin barrier integrity, mitigation of photoaging, and maintenance of overall skin health. This protective effect indicates benefits against damage induced by various types of radiation, making it relevant for both skincare and medical applications, for example as a topical formulation during cancer irradiation therapy, after radiotherapy sessions, or as a preventive treatment for exposure to X-rays, gamma rays, and other ionizing radiation.

TABLE 6
PGE2 levels after Norartocarpetin treatment

| | % of | Adjusted |
|---|---|---|
| Comparison | changes | P Value |
| UV vs. No UV | +261.79% | **** |
| UV + Norartocarpetin 1 uM vs. UV | −17.96% | ** |
| UV + Norartocarpetin 5 uM vs. UV | −74.31% | *** |

### Example 6

In another study, we measured actin organization to evaluate cytoskeletal integrity in the context of aging. Actin filaments are fundamental components of the cytoskeleton, responsible for maintaining cell shape, enabling motility, and supporting intracellular transport and signaling. Proper actin structure is essential for processes such as focal adhesion formation, mechanotransduction, and tissue repair. With aging, actin networks often become disorganized, fragmented, or less responsive to stimuli, leading to impaired cellular migration, reduced regenerative capacity, and diminished resilience against stress.

Importantly actin disassembly is linked to increased expression of TGFb and reduced synthesis of collagen (PMID: 29888864). Quantifying actin levels, distribution, and structural arrangement therefore provides a robust measure of age-related cytoskeletal decline and serves as a key indicator for assessing the efficacy of interventions aimed at preserving skin cellular architecture and function.

In this experiment, fibroblasts derived from a young donor (26 years old, Promocell, C-12302 lot 505Z006.7) and an older donor (69 years old, Promocell, C-12302 lot 495Z023.2) were analysed for actin disorganization using the Cytoskeleton kit (Sigma, ref. FAK-100). Cells were seeded on Labtek slides (ref. 154534) and treated for 24 hours with either DMSO or Norartocarpetin at 2.5 μM. Following treatment, cells were fixed and permeabilized with 0.1% Triton X-100 in 1×PBS for 1-5 minutes. After several washes, a blocking solution was applied for 30 minutes, followed by incubation with the primary antibody (anti-vinculin, 1:500 in blocking solution) for 1 hour. Cells were then washed three times and incubated with the secondary antibody for 1 hour, followed by three additional washes (5-10 minutes each) with 1× wash buffer. Nuclei were counterstained with DAPI (1:5000) for 1-5 minutes at room temperature, and cells were washed three times (5-10 minutes each) with 1× wash buffer. Slides were mounted with Fluoromount, and image analysis was conducted using ImageJ Fiji. Thresholding was applied, and particles were selected based on a size range of ≥30 and circularity values between 0.5 and 1.

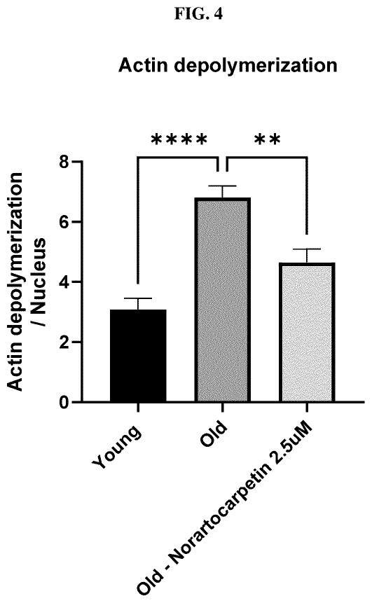

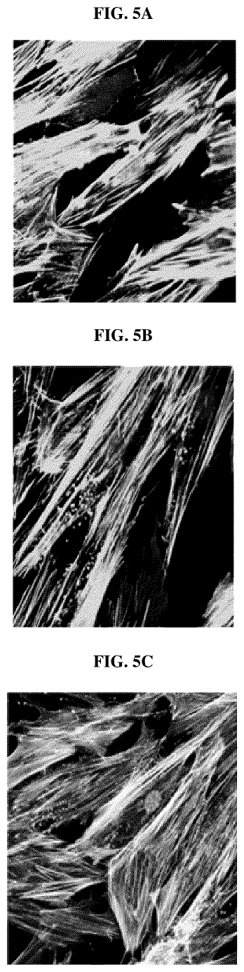

Quantitative analysis revealed that aged fibroblasts exhibited a marked increase in actin disassembly of +120.72% compared to young cells, consistent with age-associated cytoskeletal alterations. Importantly, treatment of old fibroblasts with Norartocarpetin at 2.5 μM resulted in a significant reduction of actin disorganization by −31.73% compared to untreated old cells, indicating a partial normalization of cytoskeletal structure (FIGS. 4 and 5, and Table 7). These findings demonstrate that Norartocarpetin can modulate actin dynamics, counteracting age-related disorganization and supporting maintenance of cellular architecture. This effect is thereby contributing to improved skin firmness, elasticity, and overall structural integrity, which translates into practical benefits such as enhanced resistance to wrinkle formation and maintenance of a youthful appearance

TABLE 7
Actin depolymerization

| | % of | Adjusted |
|---|---|---|
| Comparison | changes | P Value |
| Old DMSO vs. Young | +120.72% | **** |
| Old Norartocarpetin 2.5 uM vs. Old | −31.73% | ** |

### Example 7—Effect of Norartocarpetin on Nucleus Morphology

In another study, we measured nucleus area, perimeter, and circularity to evaluate cellular morphology in the context of aging. These parameters are critical indicators of nuclear integrity and function. The nucleus serves as the central hub for genetic regulation, and its size and shape directly reflect chromatin organization, transcriptional activity, and cellular health. With aging, nuclei often undergo structural changes, including enlargement, irregular contours, and loss of circularity, which are associated with genomic instability, altered gene expression, and impaired cellular resilience. Quantifying nucleus area, perimeter, and circularity therefore provides a robust means of detecting age-related nuclear alterations and assessing the efficacy of interventions aimed at preserving nuclear architecture and cellular function.

In the experiment, fibroblasts derived from a young donor (26 years old) and an old donor (69 years old) were purchase from Promocell (ref. C-12302 lot 505Z006.7 lot 495Z023.2, respectively). Cells were maintained in a low glucose DMEM (Glutamax 1g D-glucose (Gibco, ref 21885108) supplemented with FBS 10% (PanBiotech, ref P190902), 1% PenStrep (Biowest) and 1% Hepes (Biowest) until they reached 70-80% confluency. Cells were incubated at 37° C. with 5% CO2. Nuclei were stained following actin and vinculin staining using the Cytoskeleton kit (Sigma, ref. FAK-100), according to the manufacturer's instructions. Nuclei size, perimeter, and area were quantified using ImageJ by applying thresholding and selecting particles with a size ≥200.

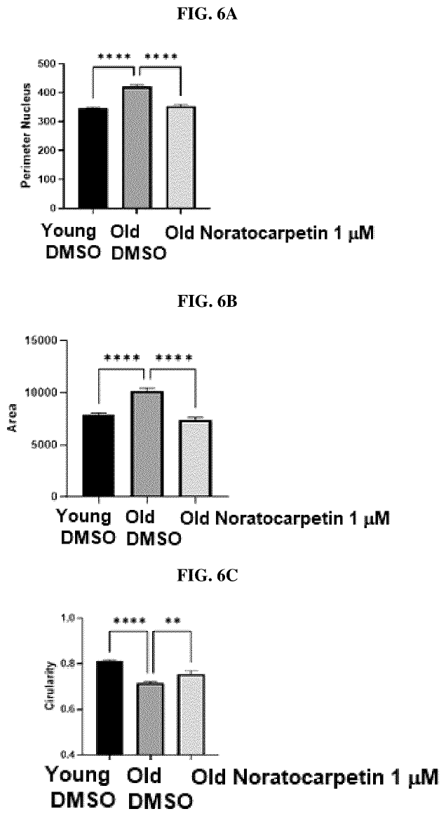

Quantitative analysis revealed that aging was associated with a +21.35% increase in nuclear perimeter, a +28.32% increase in nuclear area, and a −12.19% decrease in nuclear circularity, consistent with age-related nuclear alterations and loss of structural integrity. Importantly, treatment of old fibroblasts with Norartocarpetin at 1 μM resulted in a rescue of nuclear morphology, with perimeter reduced by −16.13%, area decreased by −27.16%, and circularity improved by +5.48% compared to untreated old cells (FIG. 6, and Tables 8, 9, 10). These findings demonstrate that Norartocarpetin can counteract age-associated nuclear disorganization, supporting the preservation of nuclear architecture and cellular function.

TABLE 8
Nucleus Perimeter

| | % of | Adjusted |
|---|---|---|
| Comparison | changes | P Value |
| Old DMSO vs. Young DMSO | +21.35% | **** |
| Old Norartocarpetin 1 uM vs. Old DMSO | −16.13% | **** |

TABLE 9
Nuclear Area

| | % of | Adjusted |
|---|---|---|
| Comparison | changes | P Value |
| Old DMSO vs. Young DMSO | +28.32% | **** |
| Old Norartocarpetin 1 uM vs. Old DMSO | −27.16% | **** |

TABLE 10
Nuclear Circularity

| | % of | Adjusted |
|---|---|---|
| Comparison | changes | P Value |
| Old DMSO vs. Young DMSO | −12.19% | **** |
| Old Norartocarpetin 1 uM vs. Old | +5.48% | **** |

### Example 8—Norartocarpetin and Resveratrol Effect on Muscle Cell Mitochondrial Respiration

The biological effects investigated in this study are related to combination of Norartocarpetin with Resveratrol.

Resveratrol is a natural polyphenol found in grapes, berries, peanuts, and red wine. It is widely studied for its antioxidant and anti-inflammatory properties, with evidence suggesting benefits for cardiovascular health, metabolism, and aging. By influencing pathways such as mitochondrial biogenesis and sirtuin activation, resveratrol has emerged as a promising compound for promoting cellular resilience and overall health.

We used the C2C12 skeletal muscle cell as a well-established model for studying mitochondrial activity due to their high energy demands. Mitochondrial responses observed in C2C12 myotubes are predictive of similar effects in other cell types, including immune, neuronal, cardiac, and epithelial cells. Therefore, findings in this model system are broadly translatable and relevant to systemic mitochondrial health.

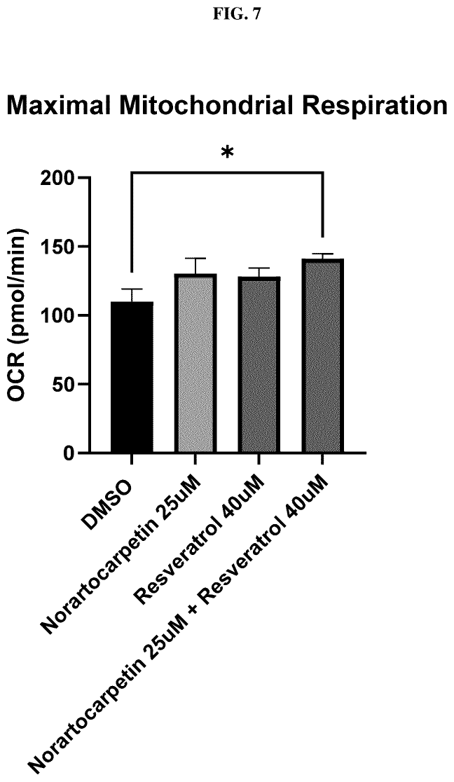

Muscle cells were treated with Norartocarpetin (25 μM) in combination with the mitochondrial biogenesis enhancer resveratrol (40 μM). As shown in Table 11, treatment with Norartocarpetin alone increased mitochondrial respiration by 18.52% compared to the DMSO control, while resveratrol alone produced an increase of 16.52% compared to DMSO. The Norartocarpetin concentration is below the normally beneficially dose, as tested in Example 1. When Norartocarpetin and Resveratrol were combined at these lower doses, mitochondrial respiration rose by 28.36%, representing a superior and statistical significant effect compared to DMSO. statistically significant improvement (FIG. 7, and Table 11). These findings indicate that while each compound individually enhances mitochondrial function to a modest degree, their combination produces an additive effect that significantly boosts respiration in muscle cells.

TABLE 11
Norartocarpetin and Resveratrol additive effects on
muscle cell mitochondrial respiration

| | % of | Adjusted |
|---|---|---|
| Comparison | changes | P Value |
| Norartocarpetin 25 uM vs.DMSO. | +18.52% | ns |
| Resveratrol 40 uM vs.DMSO | +16.52% | ns |
| Norartocarpetin 25 uM + Resveratrol | +28.36% | * |
| 40 uM vs. DMSO | | |

1. A a method of enhancing mitophagy; enhancing or maintaining mitochondrial function; treating a disease, disorder, or condition associated with a decrease in mitophagy; enhancing muscle performance; improving endurance capacity; improving, maintaining or reducing the loss of muscle function; enhancing immune health; slowing inflammaging; maintaining or enhancing brain health; or treating hair loss and/or hair thinning, comprising administering to a subject in need thereof an effective amount of one or more compounds selected from:
norartocarpetin, juglanin, meisoindigo, byakangelicol, n-nornuciferine, 3′-demethylnobiletin, 3-o-methylquercetin, dehydrocorydaline (hydroxyl), pachypodol, 4′,5-dihydroxyflavone, 5-hydroxyflavone, scutellarein tetramethyl ether, isosinensetin, robustine, skullcapflavone II, dehydrocorydaline (nitrate), coptisine, mosloflavone, iristectorin B, chrysosplenol d, phellopterin, chrysosplenetin, angelicin, and dehydrocorydaline.

2. The method of claim 1, for the treatment of a disease, disorder, or condition associated with a decrease in mitophagy, or for enhancing mitophagy.

3. The method of claim 1, wherein the subject is a generally healthy subject.

4. The method of claim 1, for enhancing or maintaining mitochondrial function.

5. The method of claim 2, wherein the wherein the disease, disorder, or condition is associated with altered mitochondrial function or reduced mitochondrial density.

6. The method of claim 2, wherein the one or more compounds increase mitochondrial activity, increase mitochondrial biogenesis, and/or to increase mitochondrial mass.

7. The method of claim 1, for enhancing muscle performance, for improving endurance capacity, or for improving, maintaining or reducing the loss of muscle function.

8. The method of claim 1, wherein the subject suffers physical fatigue, and/or muscle fatigue.

9. The method of claim 8, wherein the subject is frail or pre frail.

10. The method of claim 1, for enhancing immune health, and/or slow inflammaging.

11. The method of claim 1 for maintaining or enhancing brain health.

12. The method of claim 1 for treating or preventing skin conditions, treating hair loss and/or hair thinning.

13. A method for:
(a). treating or preventing skin inflammation;
(b). maintaining or enhancing skin health and/or appearance;
(c). maintaining or enhancing skin cell mitochondrial function;
(d). reducing or reversing skin aging;
(e). enhancing skin longevity;
(f). reducing or preventing skin wrinkles;
(g) enhancing skin healthspan;
(h) maintaining or enhancing hair health and/or appearance;
(i) treating hair loss;
(j). delaying the onset of hair loss;
(k). stimulating hair growth;
(l). increasing hair thickness;
(m). improving or maintaining hair strength;
(n). slowing or preventing premature greying of hair;
(o). healthspan and/or promoting healthy aging;
(p). treatment of a muscle-related pathological condition;
(q). a neurological disorder; and/or
(r). enhancing immune function,
comprising administering to a subject in need thereof an effective amount of one or more compounds selected from:
norartocarpetin, juglanin, meisoindigo, byakangelicol, n-nornuciferine, 3′-demethylnobiletin, 3-o-methylquercetin, dehydrocorydaline (hydroxyl), pachypodol, 4′,5-dihydroxyflavone, 5-hydroxyflavone, scutellarein tetramethyl ether, isosinensetin, robustine, skullcapflavone II, dehydrocorydaline (nitrate), coptisine, mosloflavone, iristectorin B, chrysosplenol d, phellopterin, chrysosplenetin, angelicin, and dehydrocorydaline.

14. (canceled)

15. (canceled)

16. The method of claim 13, for treatment of a muscle-related pathological condition or a neurological disorder; wherein the muscle-related pathological condition is selected from Duchenne muscular dystrophy, acute sarcopenia, and muscle atrophy and/or cachexia; and the neurological disorder is selected from cognitive decline, memory decline, anxiety, depression, epilepsy, stroke, amyotrophic lateral sclerosis, dementia, Alzheimer's disease, and Huntington disease.

17. (canceled)

18. (canceled)

19. The method of claim 13, for enhancing immune function.

20. The method of claim 1, wherein the compound is selected from Norartocarpetin, 1, Juglanin, Meisoindigo, Byakangelicol, 3′-Demethylnobiletin, 3-O-Methylquercetin, Pachypodol, 5-Hydroxyflavone, Scutellarein tetramethyl ether, and Isosinensetin.

21. The method of claim 1, wherein the compound is selected from N-Nornuciferine, Dehydrocorydaline (hydroxyl), 4′,5-Dihydroxyflavone, Robustine, Skullcapflavone II, Dehydrocorydaline (for example, the nitrate salt), Coptisine, Mosloflavone, Iristectorin B, Chrysosplenol D, Phellopterin, Chrysosplenetin, Angelicin, and Dehydrocorydaline.

22. A nutraceutical, cosmetic, or pharmaceutical composition comprising one or more compounds selected from norartocarpetin, juglanin, meisoindigo, byakangelicol, n-nornuciferine, 3′-demethylnobiletin, 3-O-methylquercetin, dehydrocorydaline (hydroxyl), pachypodol, 4′,5-dihydroxyflavone, 5-hydroxyflavone, scutellarein tetramethyl ether, isosinensetin, robustine, skullcapflavone II, dehydrocorydaline (nitrate), coptisine, mosloflavone, iristectorin B, chrysosplenol d, phellopterin, chrysosplenetin, angelicin, and dehydrocorydaline.

23. The composition of claim 22, wherein the composition is a cosmetic composition formulated for oral or topical administration.

24. The composition of claim 22, wherein the composition is a nutraceutical composition formulated for oral or topical administration.
